(19) United States
(12) Patent Application Publication
(10) Pub. No.: US 2025/0020814 A1
(43) Pub. Date: Jan. 16, 2025

(54) SCINTILLATOR CRYSTAL AND PHOTOMULTIPLIER ASSEMBLIES WITH IMPROVED EMISSION DETECTION

(71) Applicant: SEMICONDUCTOR COMPONENTS INDUSTRIES, LLC, Scottsdale, AZ (US)

(72) Inventor: Brian Patrick MCGARVEY, Templemartin (IE)

(73) Assignee: SEMICONDUCTOR COMPONENTS INDUSTRIES, LLC, Scottsdale, AZ (US)

(21) Appl. No.: 18/766,902

(22) Filed: Jul. 9, 2024

Related U.S. Application Data
(60) Provisional application No. 63/513,474, filed on Jul. 13, 2023.
(60) Provisional application No. 63/518,443, filed on Aug. 9, 2023.
(60) Provisional application No. 63/597,748, filed on Nov. 10, 2023.

(51) Int. Cl. G01T 1/20; A61B 6/03; G01T 1/29; H01L 27/146

(52) U.S. Cl. CPC G01T 1/2018; A61B 6/037; G01T 1/2985; H01L 27/14663

(57) ABSTRACT

In a general aspect, an apparatus includes a scintillator crystal, and a semiconductor die including a first side and a second side opposite the first side. The semiconductor die includes a photomultiplier array disposed on the first side. The scintillator crystal is disposed on the first side of the semiconductor die. The apparatus also includes a carrier disposed on the second side of the semiconductor die. The photomultiplier array is electrically coupled with the carrier. The apparatus further includes a molding material disposed on a sidewall defined by at least one of the semiconductor die or the carrier. The molding material is configured to protect the photomultiplier array from moisture ingress.

### CROSS-REFERENCE TO RELATED APPLICATIONS

This application claims the benefit of and priority to U.S. Provisional Application No. 63/513,474, filed Jul. 13, 2023, U.S. Provisional Application No. 63/518,443, filed Aug. 9, 2023, and U.S. Provisional Application No. 63/597,748, filed Nov. 10, 2023, all of which are incorporated by reference herein in their entireties.

### BACKGROUND

Photomultipliers are used in various systems for detecting light (photons), e.g., to perform various types of imaging. For instance, photomultipliers are used in medical imaging systems, such as positron emission tomography (PET) scanning systems (PET scanners). PET scanners can be used to evaluate function and health of organs and tissues of a patient in order to diagnose a variety of conditions, such as cancers. PET scanners operate by introducing a substance (e.g., a radiopharmaceutical, such as a radioactive glucose) into a patient's body, such as by injection or intravenous infusion. The radiopharmaceutical then interacts with tissues of the patient's body, causing the emission of gamma radiation. Diseased tissues, such as malignant tumors (or other maladies), may have greater interaction (e.g., greater uptake) of the radiopharmaceutical, resulting in higher levels of gamma radiation emission than for healthy tissues, which facilitates imaging detection of such diseased tissues. For instance, the gamma radiation is detected by one or more crystals, such as scintillator crystals, which are placed in proximity with, and surround at least a portion of the patient. The crystals then convert the detected gamma radiation to photons, which can then be detected by photomultipliers coupled with the crystals. The photomultipliers can then provide electrical signals corresponding with photon detection efficiency to an imaging system (e.g., hardware and software), which can then produce a 3-dimensional (3D) image of the body based on the detected photons, where quality of such imaging depends on efficiency of gamma radiation detection and efficiency of detection of corresponding photons.

### SUMMARY

In a general aspect, an apparatus includes a first photomultiplier having a first side including a first array of photodiodes, and a second side opposite the first side. The apparatus also includes a second photomultiplier having a first side including a second array of photodiodes, and a second side opposite the first side. The second side of the first photomultiplier is directly coupled to the second side of the second photomultiplier in a staggered arrangement.

In another general aspect, an apparatus includes a scintillator crystal, and a semiconductor die including a first side and a second side opposite the first side. The semiconductor die includes a photomultiplier array disposed on the first side. The scintillator crystal is disposed on the first side of the semiconductor die. The apparatus also includes a carrier disposed on the second side of the semiconductor die. The photomultiplier array is electrically coupled with the carrier. The apparatus further includes a molding material disposed on a sidewall defined by at least one of the semiconductor die or the carrier. The molding material is configured to protect the photomultiplier array from moisture ingress.

In another general aspect, an apparatus includes a first semiconductor die including a first photomultiplier array, and a second semiconductor die including a second photomultiplier array. The second semiconductor die is coupled in a stack with the first semiconductor die such that the first photomultiplier array is disposed at a proximal end of the stack and the second photomultiplier is disposed at a distal end of the stack. The apparatus further includes a first scintillator crystal disposed on the first semiconductor die at the proximal end of the stack, and a second scintillator crystal disposed on the second semiconductor die at the distal end of the stack. The stack is disposed between the first scintillator crystal and the second scintillator crystal. The apparatus also includes an encapsulation material disposed on a sidewall defined by the first semiconductor die and the second semiconductor die, and between the first scintillator crystal and the second scintillator crystal.

In another general aspect, an apparatus includes a scintillator crystal, a first photomultiplier disposed on a first sidewall of the scintillator crystal, and a second photomultiplier disposed on a second sidewall of the scintillator crystal. The second sidewall is orthogonal to the first sidewall. The apparatus also includes a third photomultiplier disposed on a third sidewall of the scintillator crystal, The third sidewall is parallel to the first sidewall.

### BRIEF DESCRIPTION OF THE DRAWINGS

FIG. 1A is a diagram illustrating an example photomultiplier and scintillator crystal assembly.

FIGS. 1B and 1C are diagrams respectively illustrating example conductive signals traces of photomultipliers of the assembly of FIG. 1A.

FIG. 2 is a diagram illustrating another example photomultiplier and scintillator crystal assembly.

FIGS. 3A to 3D are diagram illustrating an example process for producing back-to-back photomultipliers that can be included in the assembly of FIG. 2.

FIG. 4 is a diagram illustrating another example photomultiplier and scintillator crystal assembly.

FIGS. 5A to 5E are diagram illustrating an example process for producing back-to-back photomultipliers that can be included in the assembly of FIG. 4.

FIGS. 6A to 6C are diagrams illustrating another example process for producing back-to-back photomultipliers that can be included in the assembly of FIG. 4.

FIGS. 7A and 7B are diagrams illustrating an example photomultiplier.

FIG. 7C is a diagram illustrating an example wafer including a plurality of photomultipliers, such as the photomultiplier of FIGS. 7A and 7B.

FIGS. 8A to 8D are diagrams illustrating an example process for producing a photomultiplier and scintillator crystal assembly that can include the photomultiplier of FIGS. 7A and 7B.

FIGS. 8E and 8F are diagrams illustrating examples of alternative structures that can be produced using the process of FIG. 8A to 8D.

FIG. 9 is diagram illustrating an example photomultiplier and scintillator crystal assembly including back-to-back photomultipliers, such as the photomultiplier of FIGS. 7A and 7B.

FIG. 10A is a diagram illustrating an example single-sided photomultiplier.

FIG. 10B is a diagram illustrating an example photomultiplier and scintillator crystal assembly including the single-side photomultiplier of FIG. 10A.

FIGS. 11A to 11C are diagrams illustrating an example process for producing the photomultiplier and scintillator crystal assembly of FIG. 10B.

FIG. 12A is a diagram illustrating another example single-sided photomultiplier.

FIG. 12B is a diagram illustrating an example photomultiplier and scintillator crystal assembly including the photomultiplier of FIG. 12A.

FIGS. 13A and 13B are diagrams illustrating another example photomultiplier and scintillator crystal assembly.

FIG. 14 is a flowchart illustrating an example method for implementing the process of FIGS. 3A to 3D.

FIG. 15 is a flowchart illustrating an example method for implementing the process of FIGS. 5A to 5E.

FIG. 16 is a flowchart illustrating an example method for implementing the process of FIGS. 6A to 6C.

FIG. 17 is a flowchart illustrating an example method for implementing the process of FIGS. 8A to 8D.

FIG. 18 is a flowchart illustrating an example method for implementing the process of FIGS. 11A to 11C.

FIG. 19 is a flowchart illustrating an example method for producing the photomultiplier and scintillator crystal assembly of FIG. 12B.

In the drawings, which are not necessarily drawn to scale, like reference symbols may indicate like and/or similar components (elements, structures, etc.) in different views. The drawings illustrate generally, by way of example, but not by way of limitation, various implementations discussed in the present disclosure. Reference symbols shown in one drawing may not be repeated for the same, and/or similar elements in related views. Reference symbols that are repeated in multiple drawings may not be specifically discussed with respect to each of those drawings, but are provided for context between related views. Also, not all like elements in the drawings are specifically referenced with a reference symbol when multiple instances of an element are illustrated.

### DETAILED DESCRIPTION

This disclosure is directed to devices and assemblies that can be used in imaging systems. For instance, example assemblies or modules (imaging systems modules) including photomultiplier devices, such as silicon photomultipliers (SiPMs) and scintillation crystals are described herein. While example implementations are generally described with respect to assemblies, or modules for use in positron emission tomography (PET) scanning systems including SiPMs, in some implementations, the devices, approaches and techniques described herein can be used in assemblies or modules for other types of imaging systems, be made using different semiconductor materials, and/or can be applied in imaging systems other than PET scanners, such as computed tomography (CT) systems, or the like.

At least one technical problem associated with prior implementations of assemblies and/or modules used in, e.g., PET scanners is limitations on detection efficiency for both detection of emitted gamma radiation, e.g., by scintillator crystals, and detection of photons that are generated by gamma radiation that interacts with (intersects with, collides with, etc.) a scintillation crystal in a scanning system. Such limitations on detection efficiency for gamma radiation emitted from a patient's body during PET scan can be, at least in part, due to spacing (gaps) between scintillation crystals, where such gaps are used for implementing photomultiplier devices, printed circuit boards (PCBs) interconnecting photomultiplier device, and/or electrical connectors for providing electrical signals resulting from photon detection to external circuitry, e.g., for image processing, as some examples. Limits on photon detection efficiency can be due to locations selected for placement of photomultipliers, e.g., so as to not increase gaps between scintillator crystals, where such placement is limited in prior implementations.

Another technical problem associated with prior approaches is sensitivity to moisture ingress, which can cause various reliability issues, such as swelling of elements of an assembly, degradation (corrosion) of electrical contacts, or vias (such as through vias formed through a semiconductor die of a SiPM).

Yet another technical problem with prior approaches is limitations on reducing coincident resolution time (CRT) due, at least in part, to detection efficiencies. For instance, in PET scanning systems, radionuclides are introduced into the body. Positrons are emitted by the breakdown of the radionuclide in organs and/or tissues of the body. Gamma rays, which can be referred to as annihilation photons, are created when those emitted positrons collide with electrons near the decay event. The PET scanner then detects the gamma radiation (annihilation photons), which arrive at the detectors in coincidence at 180 degrees apart from one another, producing photons (e.g., light with a wavelength of 420 nanometer), which is detected by corresponding photomultipliers. CRT is the timing accuracy of gamma radiation detection (and corresponding photon detection) on opposite sides of the PET scanner detector array. Limitations of prior implementations for increasing detection efficiency (gamma radiation and/or resulting photons) limit improvements in CRT.

One technical solution to at least some of the aforementioned technical problems can be the use of back-to-back photomultipliers, or use of single sided photomultipliers. One technical effect of this solution is that it can allow for exclusion of elements included in prior devices and assemblies. A benefit of this technical effect can be increased gamma radiation detection efficiency, as gaps between scintillator crystals and can be reduced due to the exclusion of components between the crystals, such as exclusion of (elimination of PCBs) between crystals. Another benefit of this technical solution can be increased photon detection efficiency, as additional photomultipliers, as compared to prior approaches, can be included in an imaging system module including scintillator crystals and photomultipliers (imaging system modules, modules, etc.) without significantly increasing gaps between scintillator crystals. That is, resulting increases in photon efficiency can more than offset any effect on gamma radiation detection efficiency. Another benefit of this technical effect is improved (reduced) CRT provided by increased gamma radiation and/or photon detection.

Another technical solution to at least some of the foregoing technical problems is the use of moisture ingress prevention materials, e.g., on sidewalls of photomultiplier devices in imaging system modules. A technical effect of this technical solution can be protection of an active surfaces of photomultiplier semiconductor die (e.g., photodiode arrays, through silicon vias, etc.) from moisture ingress. One benefit of this technical effect can be prevention of moisture related reliability issues. Another technical effect of this technical solution is that it can allow for excluding or removing a glass component (cover) used, in part, for preventing moisture ingress. One benefit of this technical effect can be further reduction of gap size between scintillator crystals, which can further improve positron detection efficiency.

For purposes of illustration, the example implementations described herein are, in some instance, shown schematically in the drawings, which can be side and/or cross-sectional views. Such cross-sectional views are shown to illustrate, at least in part, structural elements of the described implementations, where such structural may be obscured, e.g., by an encapsulant or other elements, in non-sectioned views.

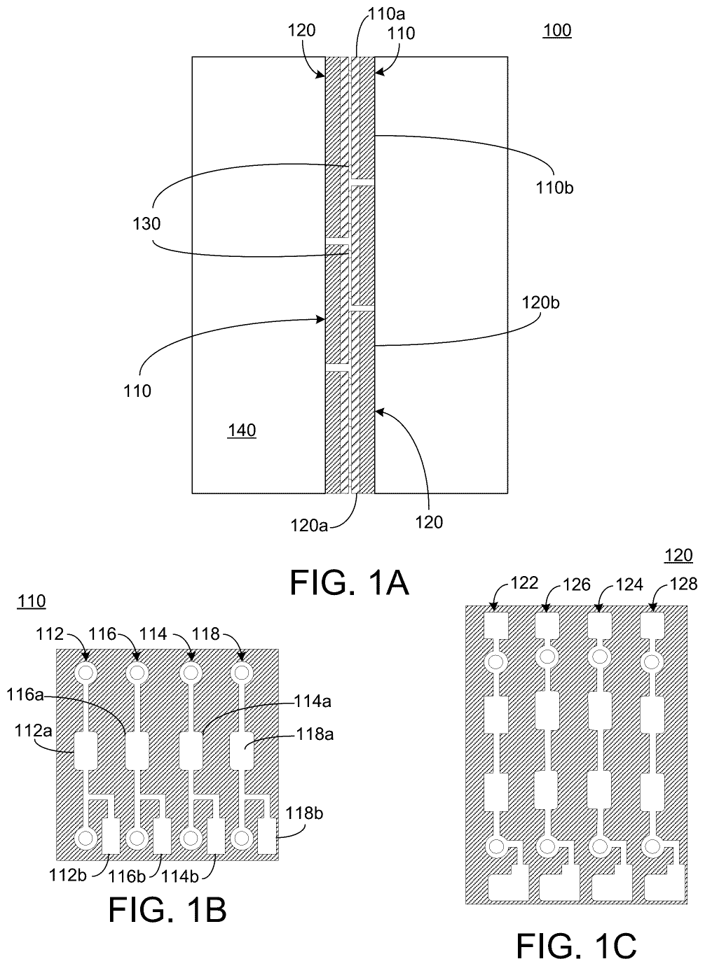

FIG. 1A is a diagram illustrating an example photomultiplier and scintillator crystal assembly (assembly 100) that can be used in an imaging system, such as a PET scanner. The assembly 100 includes a plurality of photomultipliers, e.g., photomultipliers 110 and photomultipliers 120. The photomultipliers 110 and 120 are arranged in a back-to-back staggered configuration. In this example, the photomultipliers 110 each include a photomultiplier die 110a and a glass component 110b (cover), while the photomultipliers 120 each include a photomultiplier die 120a and a glass component 120b (cover). Electrical signals generated by photodiode arrays of the photomultiplier die 110a and the photomultiplier die 120a can be conducted to backside surfaces opposite respective sides of the photomultiplier die including the photodiode arrays using through vias, e.g., through-silicon vias (TSVs). Those signals can then be communicated to external circuitry using conductive traces (copper tracks) disposed on the backsides of the photomultiplier die 110a and 120a, such as the example conductive traces shown in FIGS. 1B and 1C. Accordingly, a PCB is not used to provide signal communication for electrical signals generated by the photomultipliers 110 and 120 to external circuitry. In other words, the back-to-back, staggered arrangement of the photomultipliers 110 and 120 excludes a PCB for passing electrical signal generated by the corresponding photodiode arrays indicating detection of photons.

As shown in FIG. 1A, the assembly 100 includes scintillator crystals 140 that are respectively coupled to sides of the photomultipliers 110 and 120 that include the corresponding photodiode arrays. In some implementations the scintillator crystals 140 can be coupled with the photomultipliers 110 and 120 using an optically clear silicone adhesive to limit loss of photons due to the interface between the scintillator crystals 140 and the glass components 110b and 120b of the photomultipliers 110 and 120.

The photomultipliers 110 and 120, in this example, are coupled in the arrangement shown in FIG. 1A using solder balls 130, where the solder balls 130 of one photomultiplier can be coupled with landing pads of the conductive traces on another back-to-back, staggered photomultiplier. In some implementations, the photomultipliers 110 and 120 can be coupled in such a back-to-back, staggered configuration via wafer-to-wafer bonding of two complementary wafers, which can provide additional reduction in gap size between scintillator crystals 140. In this example, the photomultipliers 120 are larger than the photomultipliers 110, which allows for coverage (e.g., full coverage) of the sidewalls of both the scintillator crystals 140 by the back-to-back, staggered arrangement of the photomultipliers 110 and 120.

FIGS. 1B and 1C are diagrams respectively illustrating example conductive signals traces of photomultipliers 110 and 120 of the assembly 100 of FIG. 1A. For example, FIG. 1B illustrates conductive traces of a photomultiplier 110. The conductive traces of the photomultiplier 110 include a conductive trace 112 including pads 112a and 112b, a conductive trace 114 including pads 114a and 114b, a conductive trace 116 including pads 116a and 116b, and a conductive trace 118 including pads 118a and 118b. In this example, the conductive trace 112 can conduct electrical signals for anodes of the photodiode array of the photomultiplier 110, while the conductive trace 114 can conduct electrical signals for cathodes of the photodiode array. 110. In this example, as described herein, the conductive trace 112 and the conductive trace 114 can be coupled with the photodiode array of the photomultiplier 110 using through vias formed from through the photomultiplier die 110a of the photomultiplier 110, e.g., from a front side (photodiode array side) of the photomultiplier die 110a to a back side (conductive trace side) of the photomultiplier die 110a.

In this example, the conductive trace 116 and the conductive trace 118 are pass through conductive traces used to conduct electrical signals for anodes and cathodes of other photomultipliers, e.g., that are electrically coupled with the photomultiplier die 110a of the photomultiplier 110 in the back-to-back, staggered arrangement of FIG. 1A. The pads 112a, 112b, 114a, 114b, 116a, 116b, 118a and 118b can be used for electrical connections between back-to-back photomultipliers and/or connection of the photomultipliers 110 and 120, e.g., of the assembly 100, with external circuitry, such as for image processing. In some implementations, electrical connection with external circuitry can be made using a flex connector, a PCB, or other approach.

Similar to the photomultiplier 110 shown in FIG. 1B, FIG. 1C illustrates conductive traces of a photomultiplier 120 of the assembly 100 shown in FIG. 1A. The conductive traces of the photomultiplier 120 include a conductive trace 122, a conductive trace 124, a conductive trace 126, and a conductive trace 128. In this example, the conductive trace 122 can conduct electrical signals for anodes of the photodiode array of the photomultiplier 120, while the conductive trace 124 can conduct electrical signals for cathodes of the photodiode array of the photomultiplier 120. In this example, as described herein, the conductive trace 122 and the conductive trace 124 can be coupled with the photodiode array of the photomultiplier 120 using through vias formed from through the photomultiplier die 120a of the photomultiplier 120, e.g., from a front side (photodiode array side) of the photomultiplier die 120a to a back side (conductive trace side) of the photomultiplier die 120a.

In this example, the conductive trace 126 and the conductive trace 128 are pass through conductive traces used to conduct electrical signals for anodes and cathodes of other photomultipliers, e.g., that are electrically coupled with the photomultiplier die 120a of the photomultiplier 120 in the back-to-back, staggered arrangement of FIG. 1A. While not explicitly referenced in FIG. 1C, the conductive traces 122, 124, 126 and 128, as with the conductive traces of the photomultiplier 110 in FIG. 1B, include respective pads that can be used for electrical connections between back-to-back photomultipliers and/or connection of the photomultipliers 110 and 120, e.g., of the assembly 100, with external circuitry for image processing.

The conductive trace arrangements shown in FIGS. 1B and 1C are given by way of example. In some implementations, other arrangements are possible. For instance, additional conductive traces could be used, conductive trace having different layouts could be used, and so forth.

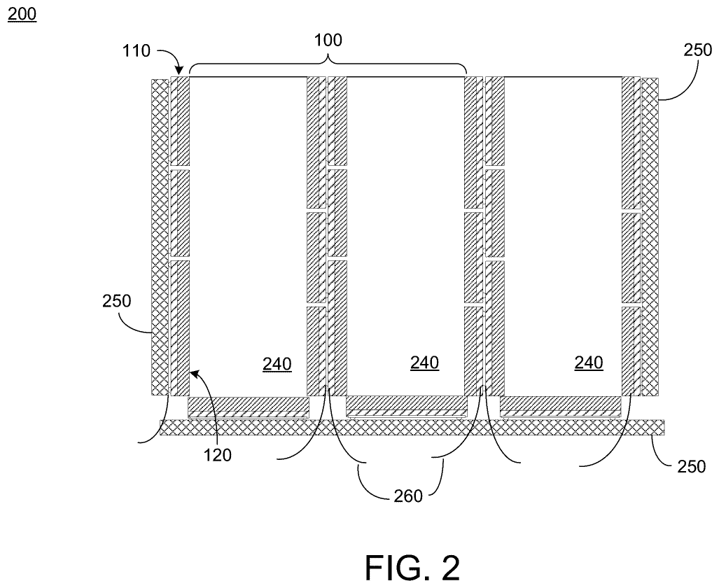

FIG. 2 is a diagram illustrating another example photomultiplier and scintillator crystal assembly (assembly 200). As shown in FIG. 2, the assembly 200 includes the assembly 100 of FIG. 1A. The assembly 200 in the example of FIG. 2 also includes another back-to-back, staggered arrangement of photomultipliers to the right of the assembly 100, which is coupled with the assembly 100 and with another scintillator crystal 240, replicating the structure of the assembly 100.

The assembly 200 also includes respective single columns of photomultipliers each including two photomultipliers 110 and one photomultiplier 120, one column on the right side of the assembly 200 and one column on the left side of the assembly 200. The assembly 200 also includes respective single photomultipliers coupled with the bottoms of the scintillator crystals of the assembly 200. In this example, back-to-back photomultiplier arrangements are not used on the left and right sides of the assembly 200 (or on the bottoms of the scintillator crystals of the assembly 200), as there are no corresponding scintillator crystals to produce photons for detection by additional, back-to-back photomultipliers.

The assembly 200 further includes PCBs on the left side, right side and bottom of the assembly 200, which can provide for signal communication between corresponding photomultipliers. Additionally, the assembly 200 includes a plurality of flex connectors, which can connect the photomultipliers of the assembly 200 with external (e.g., image processing) circuitry. The assembly 200 is configured to receive positron emissions in a PET scanner at a top of the assembly 200, where uppers sidewalls of the scintillator are exposed. In some implementations, the structure of the assembly 200 can be extended by including additional back-to-back, staggered arrangements of photomultipliers and additional, corresponding scintillator crystals.

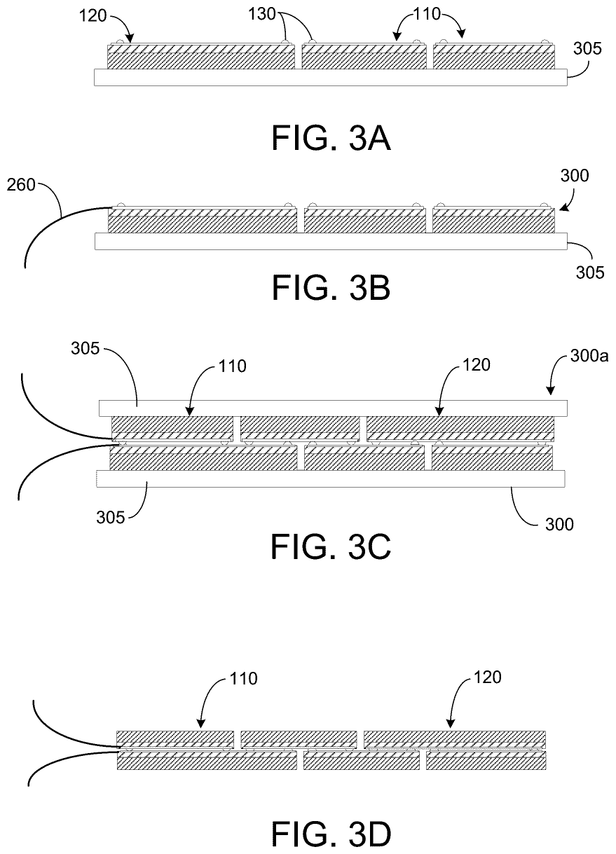

FIGS. 3A to 3D are diagram illustrating an example process for producing back-to-back photomultipliers structures that can be included in the assembly 100 of FIG. 1A and/or the assembly 200 of FIG. 2. Accordingly, for purposes of illustration, the process of FIGS. 3A to 3D is described as including elements of the assembly 100 and/or elements of the assembly 200.

As shown in 3A, a single photomultiplier 120 and two of the photomultipliers 110 are arranged on (coupled with) a carrier 305, which can be rigid, temporary carrier, such as glass, or the like. In this example, two such arrangements of photomultipliers can be produced for producing a back-to-back, staggered arrangement of photomultipliers. As shown in FIG. 3B, a flex connector 260 is coupled with the photomultiplier 120 of the structure of FIG. 3A, to produce a structure 300.

As shown in the FIG. 3C, the structure 300 of FIG. 3B can be coupled with (soldered, etc.) with a structure 300a, which is similar to the structure 300 with the exception that the flex connector 260 is coupled with one of the photomultipliers 110, rather than with the photomultiplier 120.

As shown in FIG. 3D, after coupling the structure 300 with the structure 300a, the respective carriers 305 of the structure 300 and the structure 300a can be removed. In some implementations, ultraviolet light can be used to release an adhesive used to couple the carriers 305 to their respective photo multipliers. In other implementations, heat can be moved to release an adhesive used to couple the carriers 305 to their respective photo multipliers. After the carriers 305 have been respectively removed from the structure 300 and the structure 300a, the resulting back-to-back, staggered arrangement of photomultipliers 110 and 120 of FIG. 3D can be coupled with scintillator crystals and included in an imaging system assembly, such as the assembly 100 or the assembly 200, though the structure of FIG. 3D could be included in assemblies having other configurations.

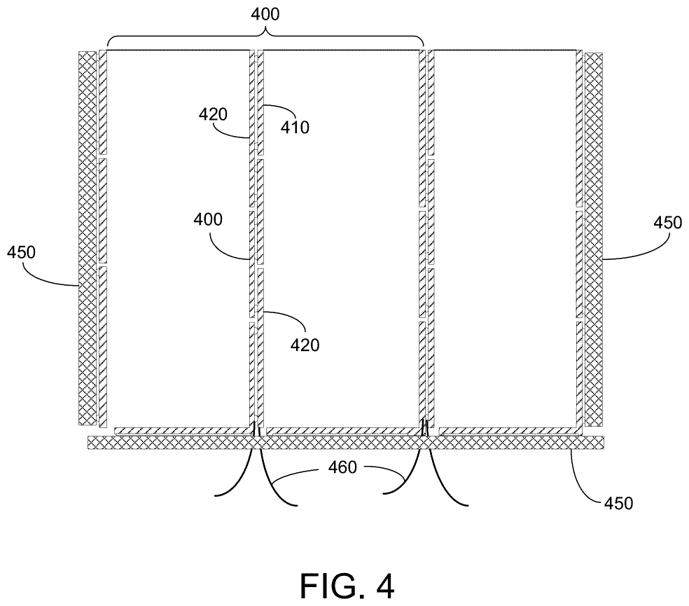

FIG. 4 is a diagram illustrating another example photomultiplier and scintillator crystal assembly (assembly). The assembly of FIG. 4 includes a module 400 including a back-to-back staggered arrangement of photomultipliers 410 and 420, The assembly of FIG. 4, similar to the assembly 200 of FIG. 2, also includes PCBs 450 and flex connectors 460. The assembly of FIG. 4 differs from the assembly 200 of FIG. 2 in that the photomultipliers (e.g., photomultipliers 410, 420 and others, such as those coupled with the bottom sidewalls of the scintillator crystals) exclude a glass cover or component. Such implementations can be advantageous as they can provide, as compared to the assembly 200 of FIG. 2, for further reduction of gaps between scintillator crystals from gaps between crystals in prior implementations where PCBs are included in a photomultiplier stack. For instance, in some implementations, a back to back stack of photomultipliers 110 can have an overall thickness that is less than 150 micrometers (μm), while each glass component (cover) can have a thickness of 400 μm. Accordingly, excluding the glass components can reduce an overall thickness of a back-to-back arrangement of photomultipliers from a thickness on the order of 0.95 millimeter (mm) to a thickness of less than 150 μm, which is greater than an eighty-four percent reduction and can result in improved positron detection efficiency.

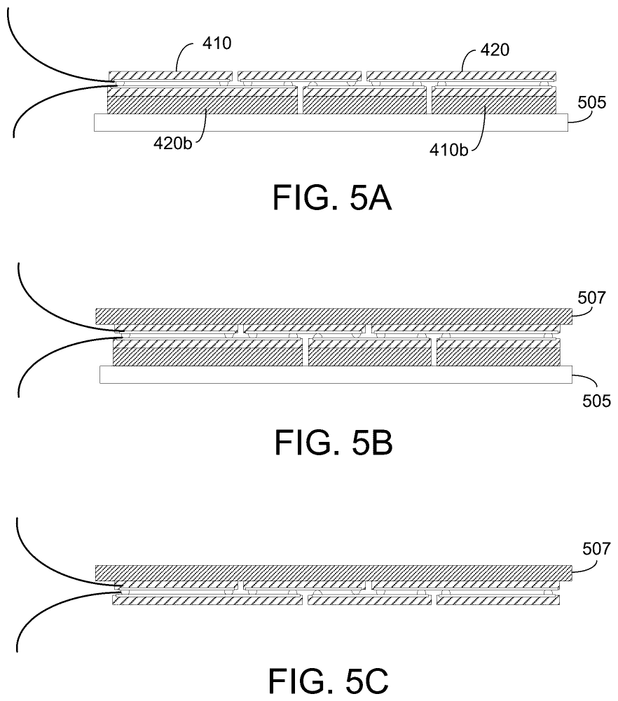

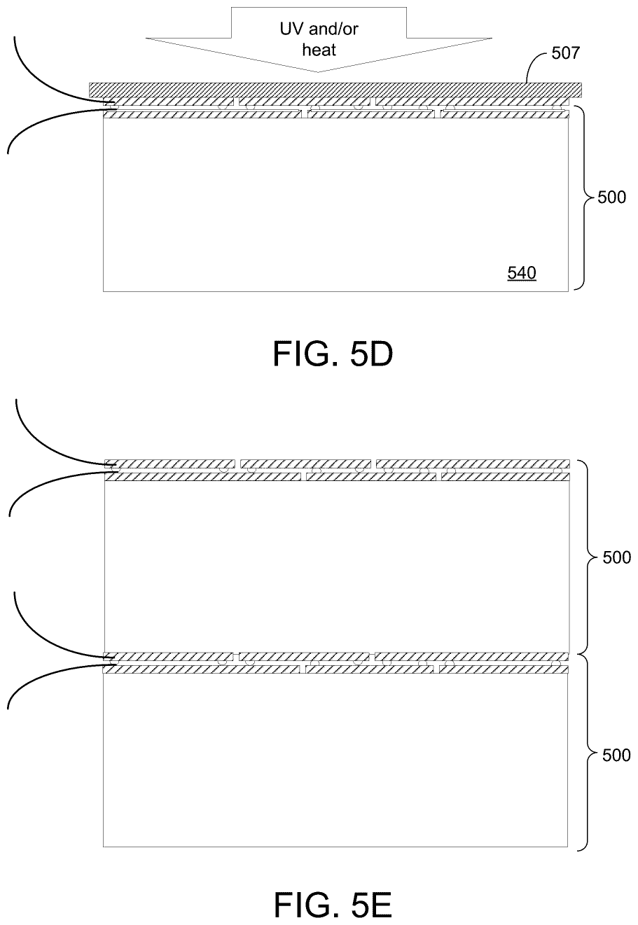

FIGS. 5A to 5E are diagrams illustrating an example process for producing back-to-back photomultipliers that can be included in, for example, the assembly of FIG. 4. In some implementations, the process of FIGS. 5A to 5E can be performed in conjunction with the operations illustrated in FIGS. 3A to 3C. For instance, the process of FIGS. 5A to 5E can be performed on structures produced using the operations illustrated in FIGS. 3A to 3C. Accordingly, for purposes of brevity, the details of those operations are not discussed in detail again with respect to process of FIGS. 5A to 5E. Also, in the discussion of FIGS. 5A to 5E, 100 series reference numbers used in FIGS. 3A to 3C are replaced with corresponding 400 series reference numbers, and 300 series reference numbers used in FIGS. 3A to 3C are replaced with 500 series reference numbers.

As shown in FIG. 5A, a carrier substrate and a glass components (covers) have been removed from one side of the structure of FIG. 3C. In some implementations, a thermal operation can be used to release the glass components from their corresponding photomultipliers. In this example, an adhesive used to couple the carrier with the glass components can be a high temperature adhesive with a higher release temperature than an adhesive used to couple the glass components with the semiconductor die of the corresponding photomultipliers 410 and 420, allowing for removal of the carrier and the glass components simultaneously.

As shown in FIG. 5B, a carrier 507 (temporary carrier) is coupled with the photomultiplier semiconductor die exposed in the operation illustrated by FIG. 5A. In example implementations, the carrier 507 can be coupled with the photomultiplier semiconductor die using an adhesive that can be released using ultraviolet light and/or heat.

As shown in FIG. 5C, the glass components 410b and 420b, along with the carrier 505 are then removed from a second (bottom) side of the structure of FIG. 5B, e.g., using ultraviolet light and/or heat. As shown in FIG. 5D, the photomultipliers on the second (bottom) side of the structure of FIG. 5C can be coupled with a scintillator crystal 540, e.g., using an optically-clear silicone adhesive to produce a structure 500, and the carrier 507 can be removed, e.g., using ultraviolet light. As shown in FIG. 5E, the structure 500 of FIG. 5D can then be coupled with another structure 500. In example implementations, the structure shown in FIG. 5E can then be used to produce an imaging system module, such as the module 400 of FIG. 4.

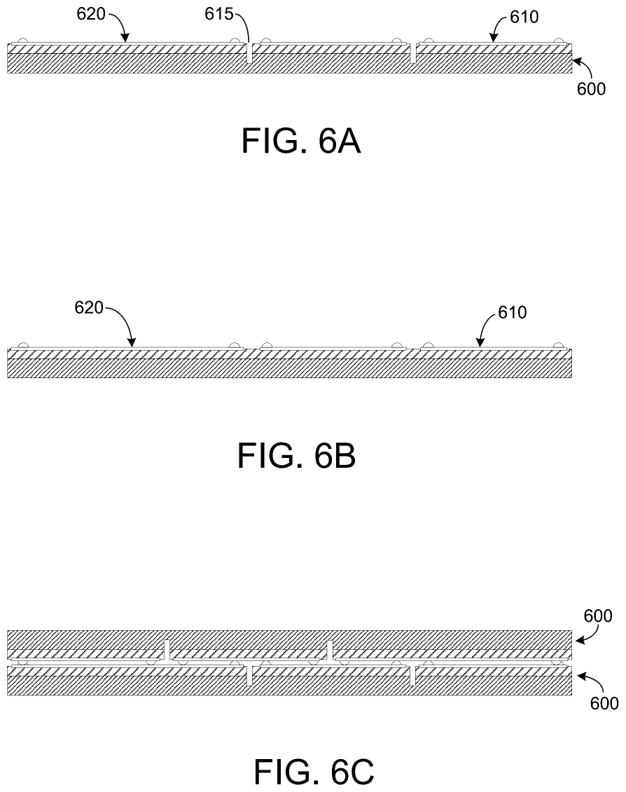

FIGS. 6A to 6C are diagrams illustrating another example process for producing back-to-back photomultipliers that can be included, for example, in the assembly of FIG. 4. The process of FIGS. 6A to 6C can be performed in conjunction with operations similar to those illustrated by FIGS. 5C to 5E, with one difference being that the carrier 507 (temporary carrier) is not used. The process of 6A to 6B can allow for provide structural and mechanical stability for photomultiplier die. As such semiconductor die can be ground to a thickness of less than 100 μm, removing the corresponding glass cover significantly increases the possibility of handling and or shipping damage, such as cracking. The process of FIGS. 6A to 6C provides additional stability, such that the respective glass covers can be removed without the use of a temporary carrier, and without significant increasing the risk of damage.

While not specifically shown in FIGS. 6A to 6C, flex connectors can be coupled with respective photomultipliers of groups of photomultipliers that are not separated. That is, as shown in FIG. 6A, in some implementations, a partial saw or laser cut operation can be performed to form openings 615 between photomultipliers 610 and 620 in a structure 600, and a flex connector can be coupled with a photomultiplier (610 or 620) of the structure 600. In some implementations, a group of photomultipliers without such openings 615, such as the structure shown in FIG. 6B, can be used to produce back-to-back, staggered arrangements of photomultipliers.

As shown in FIG. 6C, two of the structures 600 are coupled in a back-to-back configuration. In the example, respective flex connectors can be coupled with the leftmost photomultiplier in each of the top and bottom structures 600 shown in FIG. 6C. After performing a cutting operation, such as laser sawing, to fully separate the photomultipliers 610 and 620, the structure of FIG. 6C can then be further processed using operations similar to those illustrated in FIG. 5C to 5E to a produce structures, such as the structure FIG. 5E, that can be used in an imaging system module, e.g., the module 400 of FIG. 4.

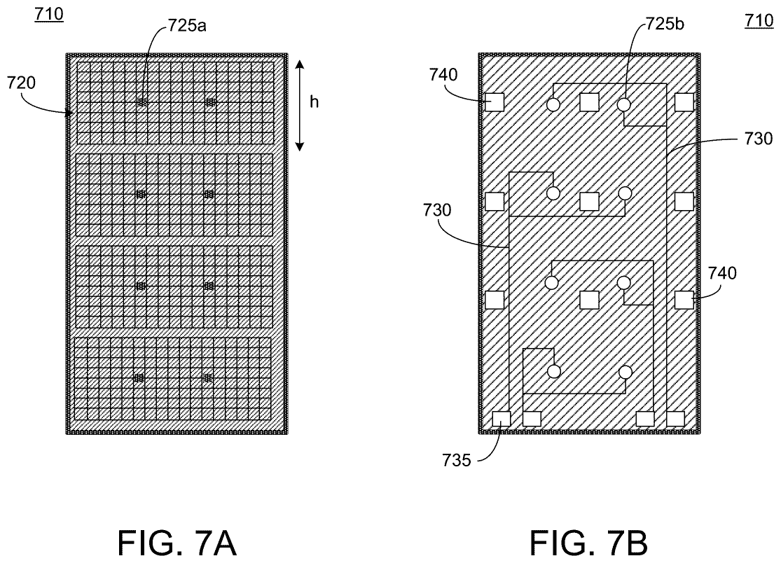

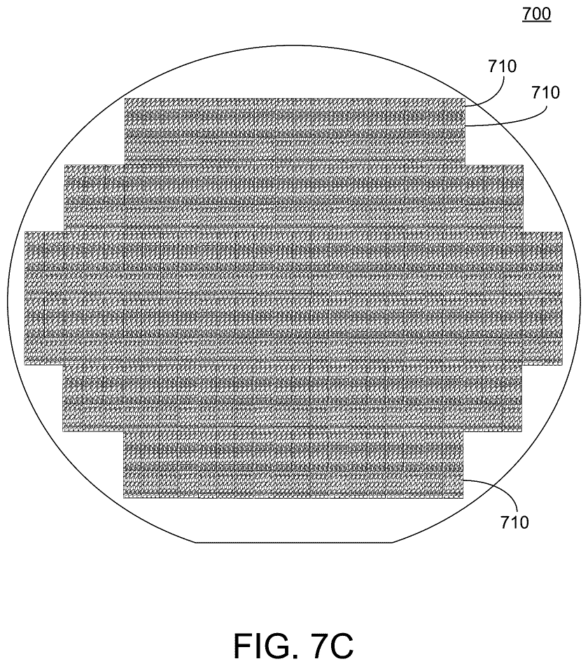

FIGS. 7A and 7B are diagrams illustrating an example photomultiplier 710. As shown in FIG. 7A, the photomultiplier 710 includes a plurality of (an array of, etc.) photomultiplier pixels 720 (micro-cells). Each photomultiplier pixels 720 of the photomultiplier 710, in this example, has a height of h, where h can be set to establish desired timing performance (e.g., coincidence resolving time) for the photomultiplier 710 in a given imaging system. Each photomultiplier pixel 720 of the photomultiplier 710 (or of other photomultipliers, such as those described herein) can include a plurality of avalanche photodiodes.

Propagation time of a signal in a photomultiplier (e.g., transit time from the photomultiplier pixels 720 to corresponding output contact pads) is dependent on h and a number of photomultiplier pixels 720. Reducing h and increasing the number of pixels, which is facilitated by the implementations described herein, will reduce signal propagation time and, as result, improve CRT.

Respective through vias having bond pads 725a on the front side (photodiode array side) of the photomultiplier 710 (FIG. 7A) can be used to connect photomultiplier pixels 720 of the photodiode array with bus lines 730 and contact pads 735 on a back side of the photomultiplier 710, where the through vias 725b are shown on the backside of the photomultiplier 710 in FIG. 7B. In some implementations, a flex connector can be electrically coupled to the contact pads 735 to connect the photomultiplier 710 with external circuitry, or the contact pads 735 electrically coupled with a carrier using wire bonds. As shown, in FIG. 7B, the back side of the photomultiplier 710 can also include electrically floating contact pads 740 for connection of the photomultiplier 710 with a rigid carrier or substrate, such as a PCB or complimentary wafer, as described below.

FIG. 7C is a diagram illustrating an example wafer 700 including a plurality of photomultipliers, such as the photomultiplier 710 of FIGS. 7A and 7B. In some implementations, the wafer 700 can be coupled with another wafer, such as another wafer 700, a passive wafer, or a wafer with different devices than the photomultiplier 710. The two wafers can be coupled using wafer-to-wafer bonding, which can produce back-to-back photomultiplier arrangements (staggered or not staggered) that can be included in imaging system assemblies of modules, such as those described herein.

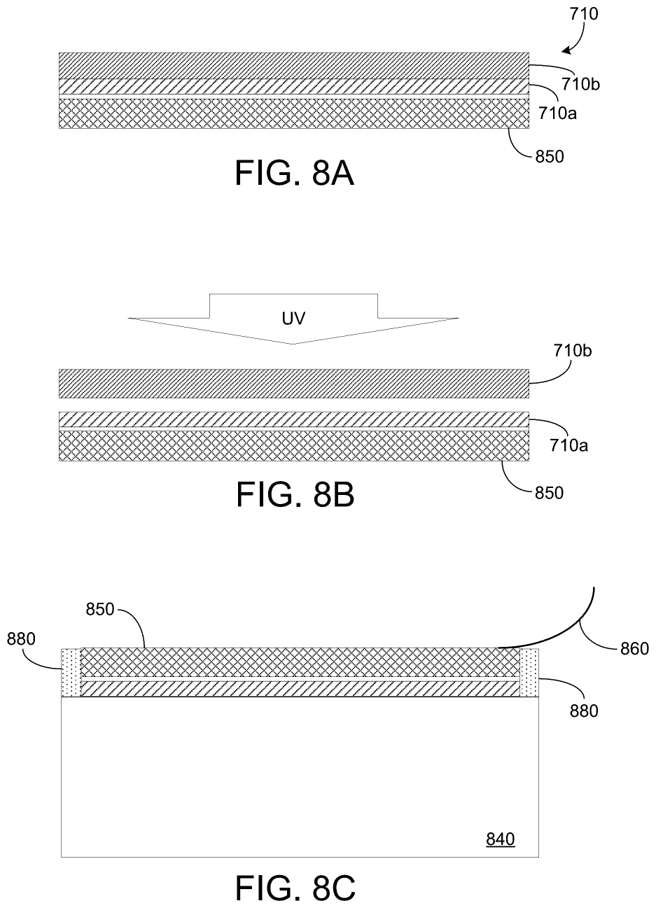

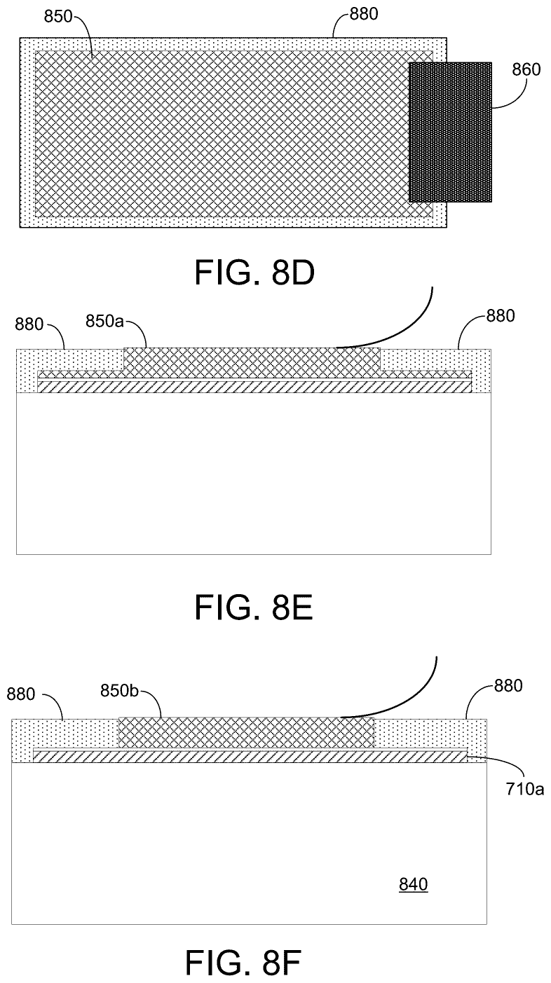

FIGS. 8A to 8D are diagrams illustrating an example process for producing a photomultiplier and scintillator crystal assembly that can include the photomultiplier 710 of FIGS. 7A and 7B. As shown in FIG. 8A, the photomultiplier 710 can be coupled with a carrier 850, such as a rigid PCB. In this example, the photomultiplier 710 includes a photomultiplier die 710a and a glass component 710b (cover). The carrier 850 can include electrical contact pads to which through vias of the photomultiplier die 710a that are electrically coupled with a photodiode array of the photomultiplier 710 can be bonded (e.g., using wafer-to-wafer bonding) or soldered (e.g., via solder balls).

As shown in FIG. 8B, the glass cover 710b can be removed from the photomultiplier die 710a, e.g., using UV light and/or heat. As shown in FIG. 8C, the photomultiplier die 710a and the carrier 850 can be coupled with a scintillator crystal 840. In this example, a stack including the photomultiplier die 710a and the carrier 850 can have an area that is smaller than an area of a corresponding sidewall of the scintillator crystal 840 to which the stack is coupled.

As further shown in FIG. 8C, a moisture ingress prevention material 880 can be formed on a sidewall defined by the photomultiplier die 710a and the carrier 850, as well as a sidewall of the scintillator crystal (840) on which the stack produced by the operation of FIG. 8B is disposed. The moisture ingress prevention material 880 can be an epoxy, silicone, or other such material. The moisture ingress prevention material 880 can be disposed (molded, formed, etc.), at least in part, around a perimeter of the sidewall defined by the photomultiplier die 710a and the carrier 850, and can prevent or reduce moisture ingress into an interface between the photomultiplier die 710a and the carrier 850. Such moisture ingress can cause reliability issues, such as failure of through vias formed in the photomultiplier die 710a. Accordingly, reducing or preventing such ingress can improve reliability over prior implementations.

As also shown in FIG. 8C, a flex connector 860 can be coupled with the carrier 850. The flex connector 860 can be used to connect the photodiode array of the photomultiplier die 710a with external circuitry, e.g., along with vias formed through the carrier 850, signal traces (e.g., signal bus lines) included in the carrier 850 and/or or the photomultiplier die 710a, and/or through vias formed in the photomultiplier die 710a. The structure of FIG. 8C can then be used to produce an imaging system module. For instance, the structure of FIG. 8C can be included in example imaging system modules, such as those described herein (e.g., the assembly 200, the module 400, as well as modules with other configurations).

FIG. 8D is a top view of the structure of FIG. 8C. As shown in FIG. 8D, the moisture ingress prevention material 880 is disposed (extends), at least in part, around a perimeter of the stack including the photomultiplier die 710a (not visible in FIG. 8D) and the carrier 850. As also shown in FIG. 8D, the flex connector 860 is coupled with the carrier 850 on a top surface of the structure of FIG. 8D (top in the view of FIG. 8C).

FIGS. 8E and 8F are diagrams illustrating examples of alternative structures that can be produced using the process of FIG. 8A to 8D. The structures of FIGS. 8E and 8F are similar to the structure of FIG. 8C, with the differences being related the shape and/or side of the carrier. For instance, in FIG. 8E, a carrier 850a, compared with the carrier 850 of FIG. 8C, is partially etched (e.g., half-etched) around its perimeter. As result, in this example, the moisture ingress prevention material 880 extends onto (over, etc.) the partially etched area. In the example of FIG. 8F, a carrier 850b has a smaller area (footprint, etc. than a sidewall of the scintillator crystal 840 on which the carrier 850 is disposed. As a result, the moisture ingress prevention material 880 extends over a portion, such as a perimeter portion of a back side surface of the photomultiplier die 710a. For example, a portion of the conductive traces on the back side of the die can be encapsulated in the moisture ingress prevention material 880.

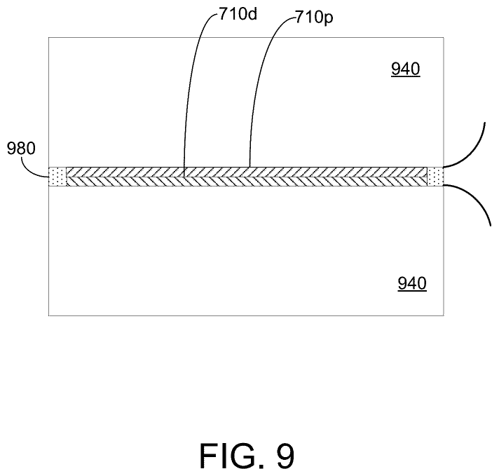

FIG. 9 is diagram illustrating an example photomultiplier and scintillator crystal assembly including back-to-back photomultipliers, such as the photomultiplier of FIGS. 7A and 7B. As shown in FIG. 9, a photomultiplier 710p is coupled back-to-back with a photomultiplier 710d, and each of the photomultipliers 710p and 710d is coupled with a respective flex connector (via contact pads 735) and a respective scintillator crystal 940 (e.g., on a photodiode array sides of the photomultipliers 710). The photomultiplier 710p (upper photomultiplier in FIG. 9) can include a photodiode array that is upward facing in FIG. 9. The photodiode array of the photomultipliers 710p can be referred to as being disposed (located, etc.) at a proximal end of the stack of the photomultiplier 710p and the photomultiplier 710d. In this example, photomultiplier 710d (lower photomultiplier in FIG. 9) can include a photodiode array that is downward facing in FIG. 9. The photodiode array of the photomultiplier 710d can be referred to as being disposed (located, etc.) a distal end of the stack of the photomultiplier 710p and the photomultiplier 710d.

As shown in FIG. 9, the respective scintillator crystals 940 are larger than the photomultipliers 710p and 710d. That is, the areas of the surfaces of the respective scintillator crystals 940 that are coupled with the photomultipliers 710p and 710d are larger than the corresponding surface areas that of the photomultipliers 710p and 710d. In this example, a moisture ingress prevention material 980 is disposed around the back-to-back arrangement of the photomultipliers 710p and 710d, and between the respective scintillator crystals 940, such as shown in FIG. 9. As discussed herein, the moisture ingress prevention material 980 provides protection again moisture ingress to the photomultipliers 710p and 710d, which can help prevent moisture related reliability issues.

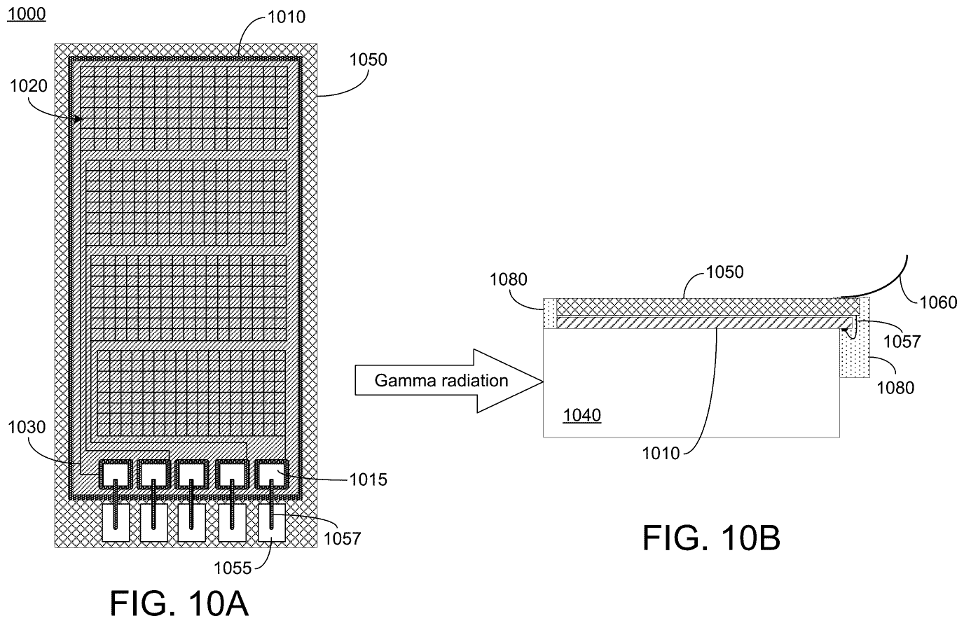

FIG. 10A is a diagram illustrating an example single-sided photomultiplier assembly 1000. Use of singled-sided photomultipliers, such as the photomultiplier die 1010, exclude the use of through vias (e.g., TSVs). Accordingly, implementations including such single-sided photomultiplier die 1010 can prevent moisture related reliability issues associated with, at least, through vias.

As shown in FIG. 10A, a photomultiplier 1010 including a plurality of photomultiplier pixels 1020 is disposed on a carrier 1050 (e.g., a rigid carrier such as a PCB). The photomultiplier die 1010 includes bus lines 1030 on the front side of the photomultiplier die 1010 (e.g., a same side as the photodiode array). The bus lines 1030 respectively connect the plurality of photomultiplier pixels 1020 with contact pads 1015. The contact pads are then electrically coupled with contact pads 1055 of the carrier 1050 via respective wire bonds 1057.

As shown in FIG. 10B, the single-sided photomultiplier assembly 1000 is coupled with a scintillator crystal 1040 in an offset arrangement. A moisture ingress prevention material 1080 is applied to (disposed on, etc.) a sidewall defined by the photomultiplier die 1010 and the sidewall of the scintillator crystal 1040 on which the stack of FIG. 10A is coupled. In this example, the moisture ingress prevention material 1080 is also applied (molded, etc.) to encapsulate the wire bonds 1057. As shown in FIG. 10B, in this example, a portion of the moisture ingress prevention material is disposed on a surface of the carrier 1050 to which the wire bonds are coupled, as well a portion of a sidewall of the scintillator crystal 1040 (e.g., a sidewall adjunct the wire bonds) and a sidewall of the carrier. The moisture ingress prevention material 1080 can be an epoxy, silicone, or other such material. The moisture ingress prevention material 1080 can prevent or reduce moisture ingress into an interface between the photomultiplier die 1010 and the carrier 1050, as well and interface between the photomultiplier die 1010 and the scintillator crystal 1040, which can reduce or prevent associated reliability issues.

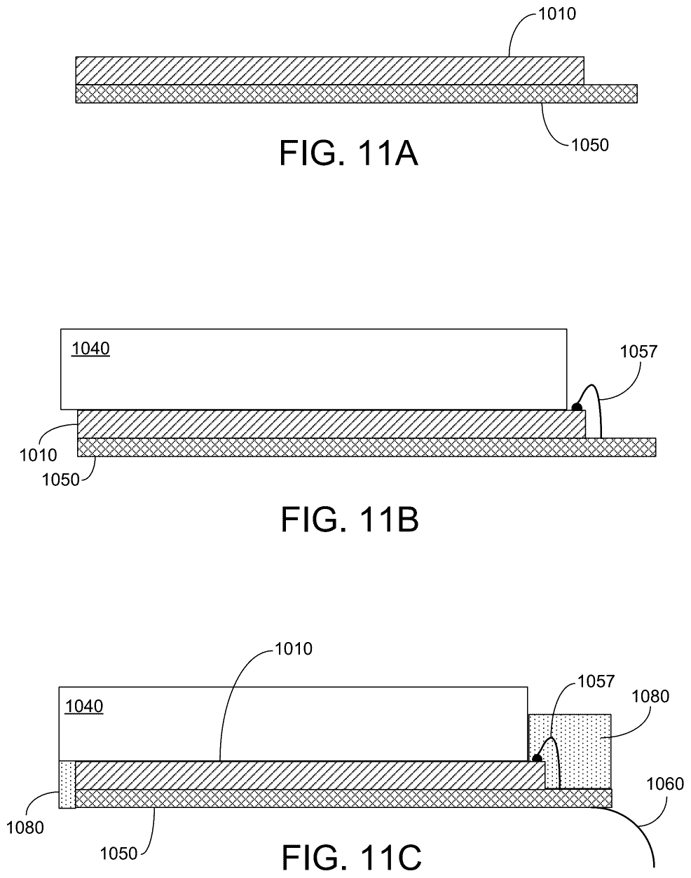

FIGS. 11A to 11C are diagrams illustrating an example process for producing the photomultiplier and scintillator crystal assembly of FIG. 10B (e.g., including the photomultiplier die 1010). As shown in FIG. 11A, in this example, the photomultiplier die 1010 is coupled with a carrier 1050, such as a rigid PCB. The carrier 1050 can include electrical contact pads 1055 to the which the photomultiplier die 1010 is electrically coupled via wire bonds 1057 (e.g., to pads 1015 of the photomultiplier die 1010). In this example, the photomultiplier 1010 does not include a glass component or cover. However, in some implementations, the operation(s) of FIG. 11A can include removing a glass component from a photomultiplier die, if present.

As shown in FIG. 11B, wire bonds 1057 are formed to electrically connect the photomultiplier die 1010 with the carrier 1050. As also shown in FIG. 11B, a stack including the photomultiplier die 1010 and the carrier 1050 are coupled with a scintillator crystal 1040. As in the example of FIG. 10B, a stack including the photomultiplier die 1010 and the carrier 1050 can have a staggered arrangement with the scintillator crystal 1040.

As shown in FIG. 11C, the moisture ingress prevention material 1080 is applied to (disposed on, etc.) a sidewall defined by the photomultiplier die 1010 and the sidewall of the scintillator crystal 1040 on which the stack of FIG. 11A is coupled. In this example, the moisture ingress prevention material 1080 is also applied (molded, etc.) to encapsulate the wire bonds 1057. As shown in FIG. 11C, in this example, a portion of the moisture ingress prevention material is disposed on a surface of the carrier 1050 to which the wire bonds are coupled, as well a portion of a sidewall of the scintillator crystal 1040 (e.g., a sidewall adjunct the wire bonds). The moisture ingress prevention material 1080 can be an epoxy, silicone, or other such material. The moisture ingress prevention material 1080 can prevent or reduce moisture ingress into an interface between the photomultiplier die 1010 and the carrier 1050, which can reduce or prevent associated reliability issues.

While not specifically shown in FIGS. 11A-11C, a flex connector can be coupled with the carrier 1050. The flex connector can be used to connect the photodiode array of the photomultiplier die 1010 with external circuitry, e.g., along with vias formed through the carrier 1050, signal traces (e.g., signal bus lines) included in the carrier 1050 and/or or the photomultiplier die 1010, and/or the wire bonds 1057. That structure can then be used to produce an imaging system module. For instance, the structure produced by process of FIGS. 11A to 11C can be included in example imaging system modules, such as those described herein (e.g., the assembly 200, the module 400, as well as modules with other configurations).

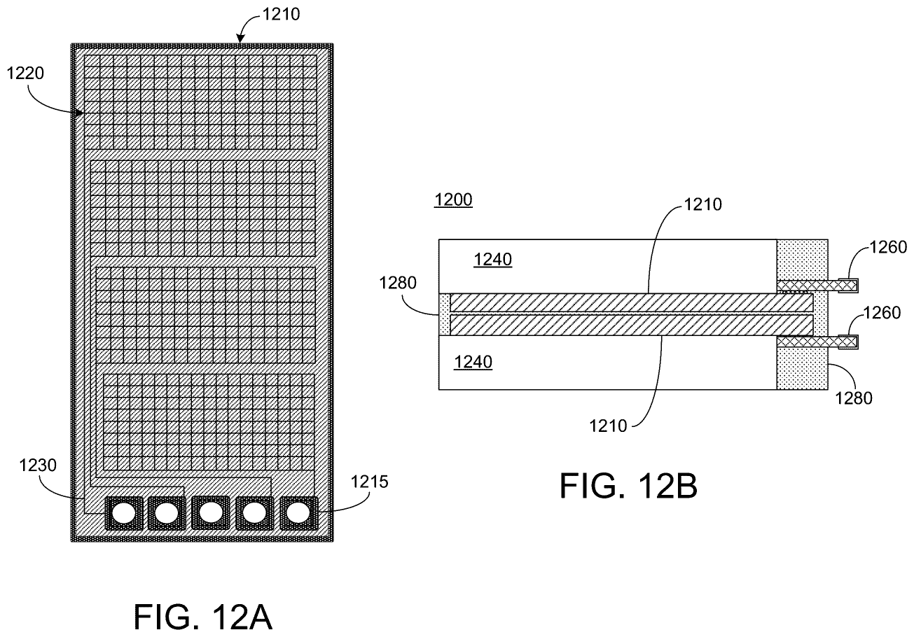

FIG. 12A is a diagram illustrating an example single-sided photomultiplier 1210. As shown in FIG. 12A, the photomultiplier 1210 includes a plurality of photomultiplier pixels 1220. The photomultiplier die 1210 includes bus lines 1230 on the front side of the photomultiplier die 1210 (e.g., a same side as the photodiode array). The bus lines 1230 respectively connect the plurality of photomultiplier pixels 1220 with contact pads 1215, which can include solder balls or an anisotropic conductive film (ACF) contact.

FIG. 12B is a diagram illustrating an example photomultiplier and scintillator crystal assembly 1200 including the photomultiplier 1210 of FIG. 12A. As shown in FIG. 12B, respective PCBs 1260 are coupled, e.g., to the contact pads 1215, of back-to-back photomultiplier die 1210. The PCBs 1260 can be coupled with the photomultiplier die using solder (e.g., solder balls), an ACF, or other approach. The photomultiplier and scintillator crystal assembly 1200 also includes respective scintillator crystals 1240 coupled with the back-to-back photomultiplier die 1210. Moisture ingress prevention material 1280 (epoxy, silicone, etc.) applied, disposed, molded, etc., such that the moisture ingress protection material 1280 contacts (covers) a sidewall defined by the stack of back-to-back photomultipliers 1210, contacts at least a portion of one more sidewalls of the scintillator crystals 1240, and partially encapsulates the PCBs 1260, where portions of the PCBs 1260 extend out of, and are external to the moisture ingress prevention material 1280, such as shown in FIG. 12B.

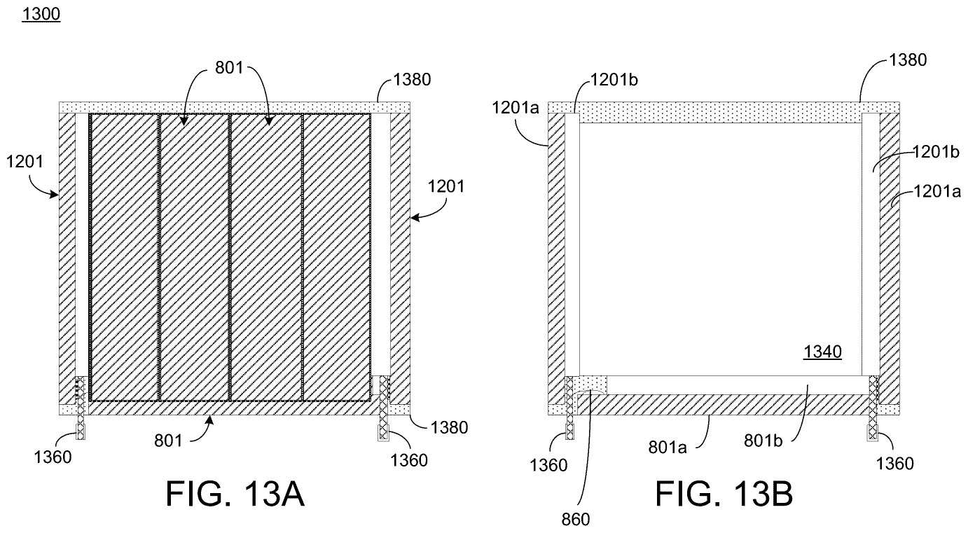

FIGS. 13A and 13B are diagrams illustrating an example of a photomultiplier and scintillator crystal assembly 1300. FIG. 13A is a side view diagram of the photomultiplier and scintillator crystal assembly 1300 and FIG. 13B is side cross-sectional view of the photomultiplier and scintillator crystal assembly 1300. The photomultiplier and scintillator crystal assembly 1300 of FIGS. 13A and 13B includes a single scintillation crystal 1340, which can be in the form a block (e.g., square cube, rectangular block, etc.) respectively having one or more photomultipliers disposed on each of a plurality of sides of the scintillation crystal 1340. For instance, in this example, the photomultiplier and scintillator crystal assembly 1300 can have photomultipliers 1201 disposed on (in the view of FIGS. 13A and 13B) on left and right sides of the scintillation crystal 1340. The photomultipliers 1201 can be similar to the photomultiplier and scintillator crystal assembly 1200 shown in FIG. 12B, but can each include only a single photomultiplier die 1201a and a single glass component 1201b, with respective PCBs 1360 coupled the respective single photomultiplier die 1201a. In some implementations, more than one photomultiplier 1201 can be included on each of the left and right sides of the scintillation crystal 1340 in the photomultiplier and scintillator crystal assembly 1300.

As also shown in FIGS. 13A and 13B, the photomultiplier and scintillator crystal assembly 1300 can include a plurality of photomultipliers 801 on (in the view of FIGS. 13A) a front side of the photomultiplier and scintillator crystal assembly 1300. The photomultipliers 801 of the photomultiplier and scintillator crystal assembly 1300 can be similar to the structure shown FIG. 8C. For instance, the photomultipliers 801 can include flex connectors 860 that can respectively connect the photomultipliers 801 to one of the PCBs 1360 and/or to external (image processing) circuitry In this example (or in other photomultiplier and scintillator crystal assemblies) respective pluralities of photomultipliers 801 can be included on a bottom side and a backside of the scintillation crystal 1340. In the views of FIGS. 13A and 13B, a single photomultiplier 801 is shown on the bottom side of the scintillation crystal 1340. Additional photomultipliers on the bottom side, and those on the backside of the scintillation crystal 1340 are obscured in these views.

In this example, the photomultiplier and scintillator crystal assembly 1300 also includes moisture ingress prevention material 1380 that is disposed, e.g., on sidewalls of components of the photomultipliers 1201 and the photomultipliers 801, such as respective sidewalls defined by photomultiplier die 801a and glass components 801b (covers), and/or respective sidewalls defined by photomultiplier die 1201a and glass components 1201b (covers). In some implementations, the photomultiplier and scintillator crystal assembly 1300 can include photomultipliers having different configuration, such as photomultipliers without glass components, back-to-back photomultiplier stacks (coupled with additional scintillation crystals), and so on.

In this example, an upper side (top side) of scintillation crystal 1340 is exposed so as to be configured to receive gamma radiation from a patient's body during a PET scan without interference. For instance, the upper side of the scintillation crystal 1340 may exclude photomultipliers as well as moisture ingress prevention material. That is, in the photomultiplier and scintillator crystal assembly 1300, photomultipliers are disposed on five of six sides of the 1340, with the sixth (top) side being exposed. In some implementations, a perimeter portion of the scintillation crystal 1340 can have moisture ingress protection material 1380 disposed thereon so as to provide protection for respective interfaces between the scintillation crystal 1340 and photomultipliers disposed on the scintillation crystal 1340. In some implementations, the perimeter portion can be a negligible portion of the total area of the top surface of the scintillation crystal 1340. For instance, in some implementations, the scintillation crystal 1340 can be a 20 mm×20 mm×20 mm cube, and the perimeter portion of the top surface of the scintillation crystal 1340 can have a width of less than 0.2 mm, such that the perimeter portion including the 1380 constitutes less than three percent of the area of the top surface of the scintillation crystal 1340.

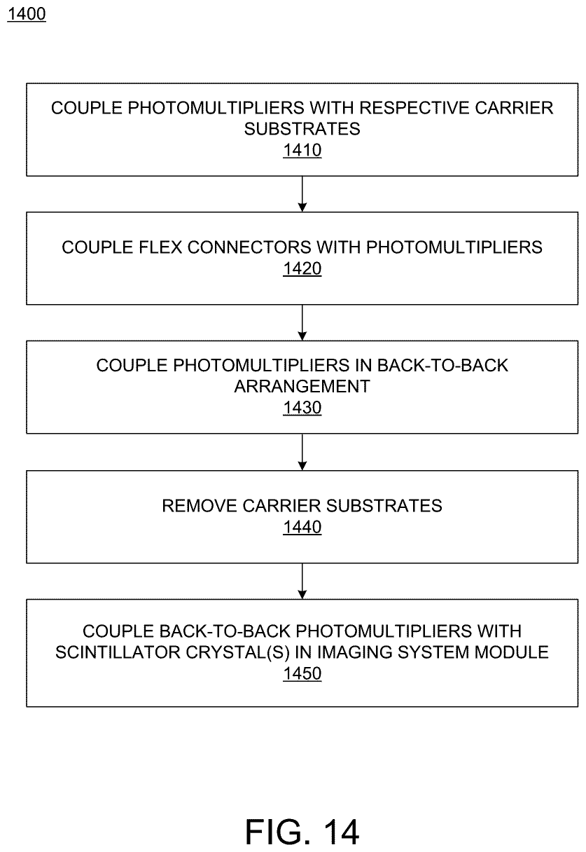

FIG. 14 is a flowchart illustrating an example method 1400 for implementing the process of FIGS. 3A to 3D. As shown in FIG. 14, at operation 1410, the method 1400 includes coupling photomultipliers with respective carrier substrates (carrier 305), such as in the arrangement shown in FIG. 3A. The carrier substrates can be temporary carriers, such as glass, ceramic, wafer tape, etc. In this example, the photomultipliers include respective semiconductor die (e.g., silicon photomultipliers) and respective glass covers (or glass components).

At operation 1420, the method 1400 includes coupling flex connectors with photomultipliers of the structures formed at operation 1410, such as in the configuration of the structure 300 shown in FIG. 3B. For instance, a first flex connector can be coupled with a photomultiplier of a first structure and a second flex connector can be coupled with a photomultiplier of a second structure, e.g., to produce two of the structures 300.

At operation 1430, the method 1400 includes coupling photomultipliers of the structures formed at operation 1420 in a back-to-back, staggered configuration, such as in the arrangement shown in FIG. 3C. In some implementations, single photomultiplier can be used to produce the structures at operation 1420. In such examples, the photomultipliers can be coupled in back-to-back, non-staggered configuration, e.g., as in the example of FIG. 9.

At operation 1440, the carrier substrate (carrier 305) on each of the structures (300) can be removed to produce the structure shown in FIG. 3D. Operation 1440 can include using ultraviolet light and/or heat to release an adhesive used to couple the carriers 305 with the photomultipliers at operation 1410.

At operation 1450, the structure(s) produced at operation 1440 can be coupled with scintillator crystals to produce an imaging system module, e.g., the assembly 200 shown in FIG. 2. In some implementations, additional photomultipliers (e.g., photomultipliers 110 and/or 120), and PCBs, such as the PCBs 250 shown in FIG. 2, can be coupled with the structures produced at operation 1450 to produce an imaging system module, such as the assembly 200.

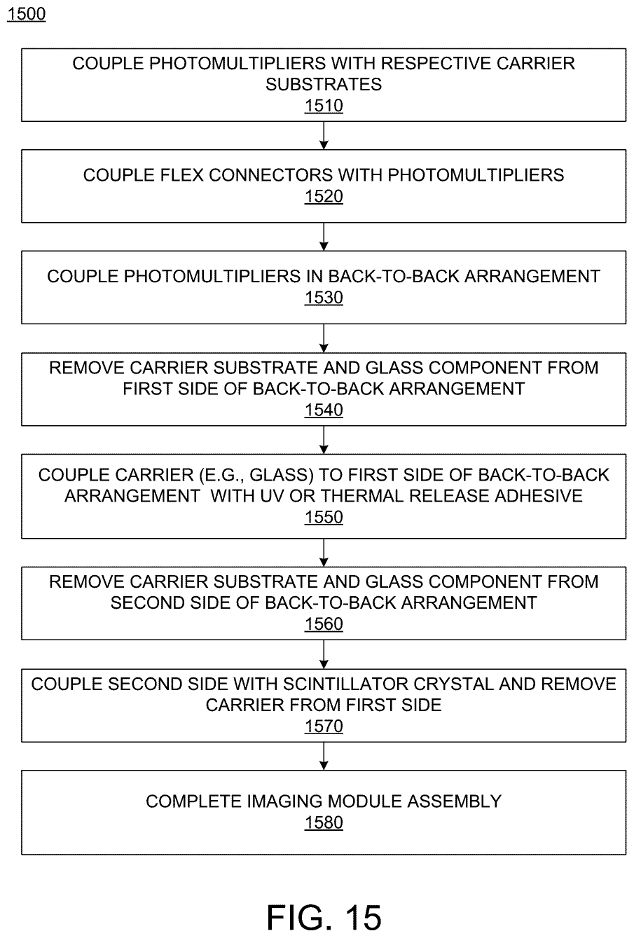

FIG. 15 is a flowchart illustrating an example method 1500 for implementing the process of FIGS. 5A to 5E. As noted above, the process of FIGS. 5A to 5E can be performed in conjunction with the operations illustrated in FIGS. 3A to 3C (e.g., the operations 1410 to 1430 of the method 1400). For instance, the operations 1510, 1520 and 1530 can be similar to, or the same as the operations 1410 to 1430 of the method 1400. Accordingly, for purposes of brevity, the details of those operations are not discussed in detail again with respect to the method 1500 of FIG. 15.

At operation 1540, the method 1500 includes removing a carrier substrate and a glass components (covers) from one of the structures coupled together at operation 1530. That is, at operation 1540, glass components 410b and 420b are removed along one of the carriers 505 from a first side of a structure, such as the structure 300 shown in FIG. 3B. In some implementations, a thermal operation can be used to release the glass components 410b and 420b from their corresponding photomultipliers. In this example, an adhesive used to couple the carriers 505 with the glass components 410b and 420b can be high temperature adhesive with a higher release temperature than an adhesive used to couple the glass components 410b and 420b with the semiconductor die of the corresponding photomultipliers, allowing for removal of the carriers 505 and the glass components 410b and 420b simultaneously.

At operation 1550, the method 1500 includes coupling a carrier (temporary carrier, such as the carrier 507 shown in FIG. 5B) with the photomultiplier semiconductor die exposed by operation 1540. In example implementations, the carrier of operation 1540 can be coupled with the photomultiplier semiconductor die using an adhesive that can be released using ultraviolet light and/or heat.

At operation 1560, the glass components 410b and 420b are removed along with the carrier 505 from a second side of the structure of produced by the operations 1540 and 1550, e.g., using ultraviolet light and/or heat. In some implementations, the operation 1560 can produce the structure shown FIG. 5C. At operation 1570, the method 1500 includes coupling the photomultipliers on the second side of the structure (e.g., the structure of operation 1560) with a scintillator crystal and removing the carrier of operation 1550, such as illustrated in FIG. 5D. At operation 1580, the structure of operation 1570 can be coupled with another scintillator crystal to produce a structure, such as the structure shown in FIG. 5E, and that structure can be used to produce an imaging system module, such as the module 400 of FIG. 4.

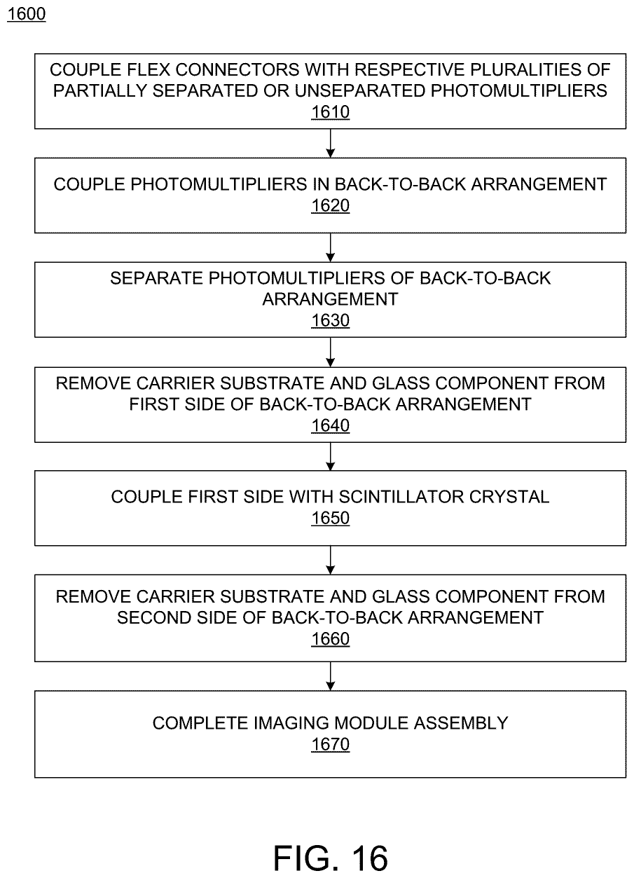

FIG. 16 is a flowchart illustrating an example method 1600 for implementing the process of FIGS. 6A to 6C. As noted above, the process of FIGS. 6A to 6C can be performed in conjunction with the operations similar to those illustrated by FIGS. 5C to 5E, e.g., the operations 1560 to 1580 of the method 1500. For instance, the operations 1640 to 1670 of the method 1600 can be similar to the operations shown by FIGS. 5C to 5E and as discussed with respect to FIG. 15, with one difference being that a temporary carrier (of operation 1650) is not used in the method 1600.

At operation 1610, the method 1600 includes coupling flex connectors with respective photomultipliers of groups of photomultipliers that are not separated. That is, as shown in FIG. 6A, in some implementations, a partial saw or laser cut operation can be performed to form openings 615 between photomultipliers 610 and 620 in a structure 600. In some implementations, a group of photomultipliers without such openings 615 can be used, as shown in FIG. 6B. In the example of FIGS. 6A to 6C, flex connectors are not explicitly shown, but can be included (e.g., coupled with the photomultiplier 620) as in other example implementations described herein.

At operation 1620, the method 1600 includes coupling the structures of operation 1610 in a back-to-back configuration, such as the staggered (interleaved) configuration of FIG. 6C. At operation 1630, the photomultipliers of the structure of FIG. 6C can be fully separated, e.g., laser sawn to fully separate the photomultipliers (e.g., semiconductor die and corresponding glass components). At operation 1640, a carrier substrate and the glass components can be removed from a first side of the structure of FIG. 6C (after separation an operation 1630), similar to the operation of FIG. 5C. At operations 1650 and 1660, a scintillator crystal can be coupled with the photomultiplier die exposed at operation 1640 (operation 1650) and the carrier substrate and glass components can be removed from a second side of the structure of FIG. 6C (operation 1660), similar to the operation of FIG. 5D. At operation 1670, the structure of operation 1660 can be coupled with another scintillator crystal to produce, e.g., the structure shown in FIG. 5E, and that structure can be used to produce an imaging system module, such as the module 400 of FIG. 4.

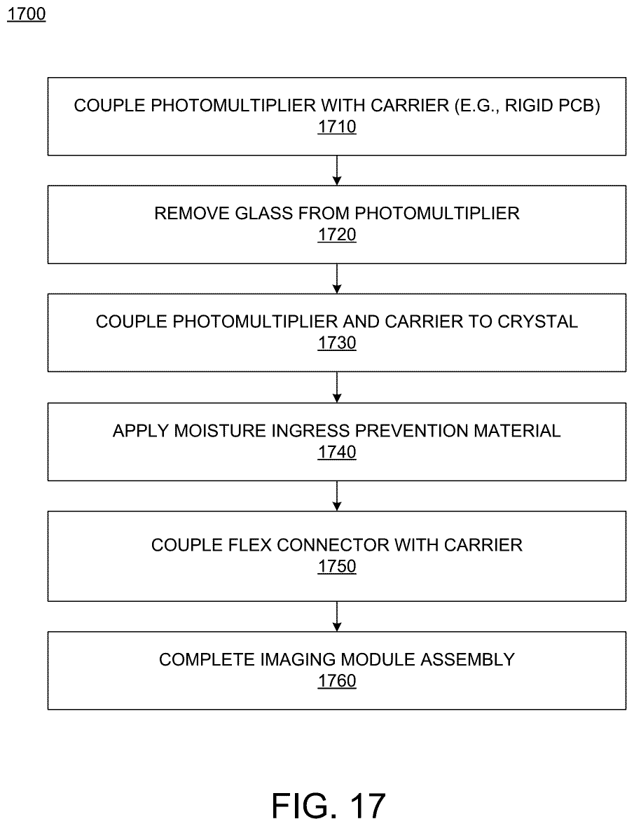

FIG. 17 is a flowchart illustrating an example method 1700 for implementing the process of FIGS. 8A to 8C, and/or to produce the example structures of FIGS. 8C to 8F. At operation 1710, the method 1700 includes coupling a photomultiplier (semiconductor die and glass cover) with a carrier, such as a rigid PCB. The carrier can include electrical contact pads to which through vias of the semiconductor die that are electrically coupled with a photodiode array of the photomultiplier can be bonded (e.g., using wafer-to-wafer) or soldered (e.g., via solder balls). The operation 1710 can produce a structure such as that shown in FIG. 8A.

At operation 1720, the method 1700 includes removing a glass cover (glass component) from the photomultiplier, e.g., using UV light and/or heat, such as illustrated in FIG. 8B. At operation 1730, the method 1700 includes coupling the photomultiplier die and carrier of operation 1720 with a scintillator crystal (840), such as shown in FIG. 8C. As in the example of FIG. 8C, a stack including photomultiplier die (710a) and the carrier (850) can have an area that is smaller than a corresponding sidewall of the scintillator crystal (840) to which the stack is coupled.

The method 1700 then includes, at operation 1740, applying a moisture ingress prevention material (880) to a sidewall defined by the photomultiplier die (710a) and the sidewall of the scintillator crystal (840) on which the stack produce at operation 1720 is coupled. The moisture ingress prevention material (880) can be an epoxy, silicone, or other such material. The moisture ingress prevention material (880) can be disposed, at least in part, around a perimeter of a sidewall defined by the photomultiplier die (710a) and the carrier (850) and can prevent or reduce moisture ingress into an interface between the photomultiplier die (710a) and the carrier (850). Such moisture ingress can cause reliability issues, such as failure of through vias formed in the photomultiplier die 710a. Accordingly, reducing or preventing such ingress can improve reliability over prior implementations.

At operation 1750, the method includes coupling a flex connector (860) with the carrier 850, as also shown in FIG. 8C. The flex connector can be used to connect the photodiode array of the photomultiplier die (710a) with external circuitry, e.g., along with vias formed through the carrier (850), signal traces (e.g., signal bus lines) included in the carrier (850) and/or or the photomultiplier die (710a), and/or through vias formed in the photomultiplier die (710a). At operation 1760, the method 1700 includes completing an imaging system module including the structure produced by the foregoing operations. For instance, the structure produced by the method 1700 can be included in example imaging system modules, such as those described herein (e.g., the assembly 200, the module 400, as well as modules with other configurations).

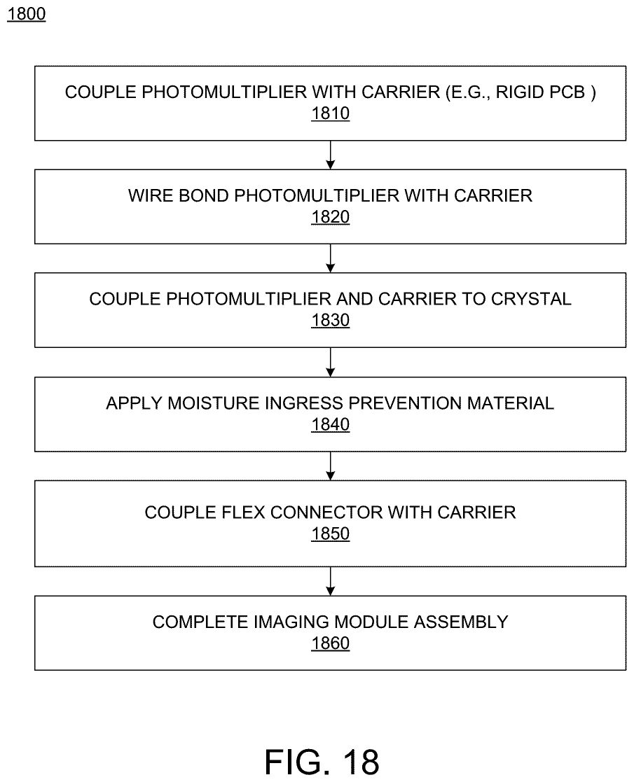

FIG. 18 is a flowchart illustrating an example method 1800 for implementing the process of FIGS. 11A to 11C. At operation 1810, the method 1800 includes coupling a photomultiplier die 1010 with a carrier, such as a rigid PCB (carrier 1050). The carrier can include electrical contact pads to the which the photomultiplier die 1010 is electrically coupled via wire bonds. The operation 1810 can produce a structure such as that shown in FIG. 11A. In this example, the photomultiplier does not include a glass component or cover. In some implementations, the operation 1810 can include removing a glass component from a photomultiplier die, if present.

At operation 1820, the method 1800 includes forming wire bonds to electrically connect the photomultiplier with the carrier. At operation 1830, the method 1800 includes coupling the photomultiplier die and carrier of operation 1820 with a scintillator crystal (1040), such as shown in FIG. 10B. As in the example of FIG. 10B, a stack including the photomultiplier die (1010) and the carrier (1050) can have a staggered arrangement with the scintillator crystal 1040.

The method 1800 then includes, at operation 1840, applying a moisture ingress prevention material (1080) to a sidewall defined by the photomultiplier die (1010) and the sidewall of the scintillator crystal (1040) on which the stack produced at operation 1810 is coupled. In this example, the moisture ingress prevention material (1080) is also applied (molded, etc.) to encapsulate the wire bonds (1057). As shown in FIG. 11C, in this example, a portion of the moisture ingress prevention material is disposed on a surface of the carry to which the wire bonds are coupled, as well a portion of a sidewall of the scintillator crystal. The moisture ingress prevention material can be an epoxy, silicone, or other such material. The moisture ingress prevention material can prevent or reduce moisture ingress into an interface between the photomultiplier die and the carrier, which can reduce or prevent associated reliability issues.

At operation 1850, the method 1800 includes coupling a flex connector (1060) with the carrier, as also shown in FIG. 11C. The flex connector can be used to connect the photodiode array of the photomultiplier die with external circuitry, e.g., along with vias formed through the carrier, signal traces (e.g., signal bus lines) included in the carrier and/or or the photomultiplier die, and/or the wire bonds. At operation 1860, the method 1800 includes completing an imaging system module including the structure produced by the foregoing operations. For instance, the structure produced by the method 1800 can be included in example imaging system modules, such as those described herein (e.g., the assembly 200, the module 400, as well as modules with other configurations).

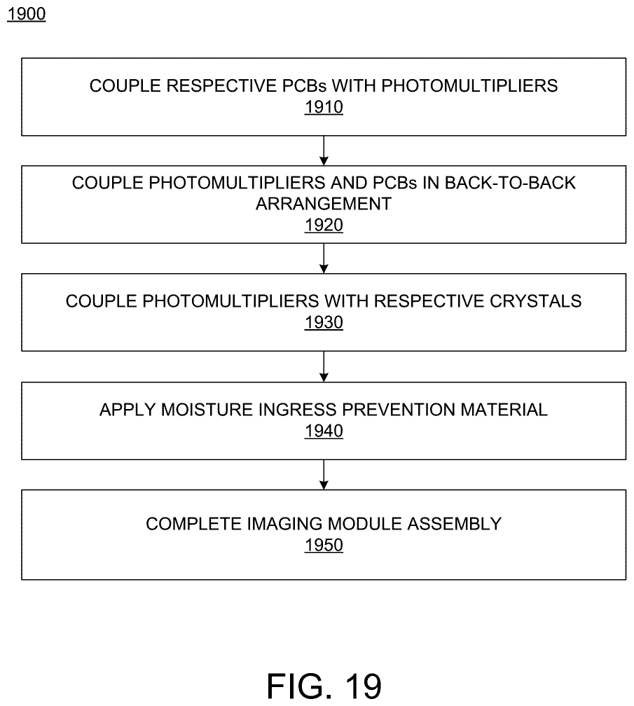

FIG. 19 is a flowchart illustrating an example method 1900 for producing the photomultiplier and scintillator crystal assembly of FIG. 12B. At operation 1910, the method 1900 includes coupling respective PCBs with photomultiplier die. The PCBs can be coupled with the photomultiplier die using solder (e.g., solder balls), an anisotropic conductive film (ACF), or other approach. At operation 1920, the method 1900 includes coupling the photomultiplier and PCB structures of operation 1910 in a back-to-back configuration, such as shown in FIG. 12B. At operation 1930, the method 1900 includes coupling the photomultipliers with respective scintillator crystals. At operation 1940, moisture ingress prevention material (epoxy, silicone, etc.) can be applied such that the moisture ingress protection material contacts (covers) a sidewall defined by the stack of back-to-back photomultipliers, contacts at least a portion of one more sidewalls of the scintillator crystals, and partially encapsulates the PCBs, where portions of the PCBs extend out of, and are external to the moisture ingress prevention material, such as shown in FIG. 12B. At operation 1950, the structure produced by operations 1910 to 1940 can be used to produce an imaging system module, such as the imaging system module 1300 of FIGS. 13A and 13B.

In a general aspect, an apparatus includes a first photomultiplier having a first side including a first array of photodiodes, and a second side opposite the first side. The apparatus also includes a second photomultiplier having a first side including a second array of photodiodes, and a second side opposite the first side. The second side of the first photomultiplier is directly coupled to the second side of the second photomultiplier in a staggered arrangement.

Implementations can include one or more of the following features or aspects, alone or in combination. For example, the second side of the first photomultiplier can be soldered to the second side of the second photomultiplier.

A printed circuit board can be excluded from a stack including the first photomultiplier and the second photomultiplier.

The first photomultiplier can include at least one cathode terminal configured to be connected to external circuitry, and at least one anode terminal configured to be connected to the external circuitry.

The first photomultiplier can be included in a first wafer and the second photomultiplier can be included in a second wafer. The first wafer can be wafer bonded to the second wafer.

The apparatus can include a scintillator crystal coupled with the first side of the first photomultiplier. The apparatus can be configured for use in a positron emission tomography (PET) scanner.

The scintillator crystal can be a first scintillator crystal. The apparatus can include a second scintillator crystal coupled with the first side of the second photomultiplier.

The first photomultiplier can include a first plurality of conductive traces disposed on the second side of the first photomultiplier, and a first plurality of through vias respectively electrically coupling a first portion of the first plurality of conductive traces with the first array of photodiodes. The second photomultiplier can include a second plurality of conductive traces disposed on the second side of the second photomultiplier, and a second plurality of through vias respectively electrically coupling a first portion of the second plurality of conductive traces with the second array of photodiodes.

A second portion of the first plurality of conductive traces can be respectively electrically coupled with the first portion of the second plurality of conductive traces. A second portion of the second plurality of conductive traces can be respectively electrically coupled with the first portion of the first plurality of conductive traces.

The apparatus can include a flex connector coupled with the first photomultiplier. The flex connector can be configured to connect the first photomultiplier with external circuitry.

The first photomultiplier can include a first semiconductor die including the first array of photodiodes, and a first glass cover disposed on the first array of photodiodes. The second photomultiplier can include a second semiconductor die including the second array of photodiodes, and a second glass cover disposed on the second array of photodiodes.

The first photomultiplier and the second photomultiplier can each exclude a glass cover.

In another general aspect, an apparatus includes a scintillator crystal, and a semiconductor die including a first side and a second side opposite the first side. The semiconductor die includes a photomultiplier array disposed on the first side. The scintillator crystal is disposed on the first side of the semiconductor die. The apparatus also includes a carrier disposed on the second side of the semiconductor die. The photomultiplier array is electrically coupled with the carrier. The apparatus further includes a molding material disposed on a sidewall defined by at least one of the semiconductor die or the carrier. The molding material is configured to protect the photomultiplier array from moisture ingress.

Implementations can include one or more of the following features or aspects, alone or in combination. For example, the carrier can be electrically coupled with the photomultiplier array by at least one via that extends through the semiconductor die from the first side of the semiconductor die to the second side of the semiconductor die.

The carrier can be electrically coupled with the photomultiplier array by at least one wire bond encapsulated in the molding material.

The apparatus can include a flex connector coupled with the carrier. The flex connector can be configured to connect the photomultiplier array with external circuitry.

The molding material can include at least one of epoxy or silicone.

The molding material can extend over at least a portion of the second side of the semiconductor die.

A perimeter portion of the carrier can be partially etched. The molding material can extend over the partially etched perimeter portion of the carrier.

In another general aspect, an apparatus includes a first semiconductor die including a first photomultiplier array, and a second semiconductor die including a second photomultiplier array. The second semiconductor die is coupled in a stack with the first semiconductor die such that the first photomultiplier array is disposed at a proximal end of the stack and the second photomultiplier is disposed at a distal end of the stack. The apparatus further includes a first scintillator crystal disposed on the first semiconductor die at the proximal end of the stack, and a second scintillator crystal disposed on the second semiconductor die at the distal end of the stack. The stack is disposed between the first scintillator crystal and the second scintillator crystal. The apparatus also includes an encapsulation material disposed on a sidewall defined by the first semiconductor die and the second semiconductor die, and between the first scintillator crystal and the second scintillator crystal.

Implementations can include one or more of the following features or aspects, alone or in combination. For example, the apparatus can include a first flex connector coupled with the first semiconductor die, The first flex connector can be configured to connect the first photomultiplier array with external circuitry. The apparatus can include a second flex connector coupled with the second semiconductor die. The second flex connector can be configured to connect the second photomultiplier array with the external circuitry.

The first semiconductor die can be directly coupled with second semiconductor die.

The first scintillator crystal can be directly coupled with the first semiconductor die with an optically clear adhesive. The second scintillator crystal can be directly coupled with the second semiconductor die with the optically clear adhesive.

In another general aspect, an apparatus includes a scintillator crystal, a first photomultiplier disposed on a first sidewall of the scintillator crystal, and a second photomultiplier disposed on a second sidewall of the scintillator crystal. The second sidewall is orthogonal to the first sidewall. The apparatus also includes a third photomultiplier disposed on a third sidewall of the scintillator crystal, The third sidewall is parallel to the first sidewall.

Implementations can include one or more of the following features or aspects, alone or in combination. For example, the apparatus can include at least one glass component respectively disposed between at least one of the first photomultiplier and the first sidewall, the second photomultiplier and the second sidewall, or the third photomultiplier and the third sidewall.

The second sidewall is on a side of the scintillator crystal opposite a fourth sidewall of the scintillator crystal configured to receive gamma radiation in a positron emission tomography scanner.

A first printed circuit board (PCB) can be electrically coupled to the first photomultiplier. A second PCB can be electrically coupled to the third photomultiplier. The third photomultiplier can be electrically coupled to the first PCB via a flex connector.

The apparatus can include a fourth photomultiplier disposed on a fourth sidewall of the scintillator crystal. The fourth sidewall can be orthogonal to the first sidewall, the second sidewall and the third sidewall. The apparatus can include a fifth photomultiplier disposed on a fifth sidewall of the scintillator crystal. The fifth sidewall being orthogonal to the first sidewall, the second sidewall and the third sidewall, and parallel to the fourth sidewall.

The apparatus can include a molding material encapsulating at least a portion of the scintillator crystal and being in contact with, at least, respective portions of the first photomultiplier, the second photomultiplier, and the third photomultiplier, such that at least a fourth sidewall of the scintillator crystal is exposed through the molding material.

A number of implementations have been described. Nevertheless, it will be understood that various modifications may be made without departing from the spirit and scope of the specification.

It will also be understood that when an element is referred to as being on, connected to, electrically connected to, coupled to, or electrically coupled to another element, it may be directly on, connected or coupled to the other element, or one or more intervening elements may be present. In contrast, when an element is referred to as being directly on, directly connected to or directly coupled to another element, there are no intervening elements or layers present. Although the terms directly on, directly connected to, or directly coupled to may not be used throughout the detailed description, elements that are shown as being directly on, directly connected or directly coupled can be referred to as such. The claims of the application may be amended to recite illustrative relationships described in the specification or shown in the figures.

The various apparatus and techniques described herein may be implemented using various semiconductor processing and/or packaging techniques. Some implementations may be implemented using various types of semiconductor processing technologies associated with semiconductor substrates including, but not limited to, for example, silicon (Si), gallium arsenide (GaAs), silicon carbide (SiC), gallium nitride (GaN), and/or so forth.

As used in this specification, a singular form may, unless definitely indicating a particular case in terms of the context, include a plural form. Spatially relative terms (e.g., over, above, upper, under, beneath, below, lower, and so forth) are intended to encompass different orientations of the device in use or operation in addition to the orientation depicted in the figures. In some implementations, the relative terms above and below can, respectively, include vertically above and vertically below. In some implementations, the term adjacent can include laterally adjacent to or horizontally adjacent to.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the implementations. It should be understood that they have been presented by way of example only, not limitation, and various changes in form and details may be made. Any portion of the apparatus and/or methods described herein may be combined in any combination, except mutually exclusive combinations. The implementations described herein can include various combinations and/or sub-combinations of the functions, components and/or features of the different implementations described.

In addition, the logic and/or process flows depicted in the figures do not require the particular order shown, or sequential order, to achieve desirable results. In addition, other operations may be included, or operations may be eliminated, from the described flows, and other components or elements may be added to, or removed from the described devices, methods and/or systems. Accordingly, other implementations are within the scope of the following claims.

It will be understood that, although the terms first, second, etc. may be used herein to describe various elements, these elements should not be limited by these terms. These terms are only used to distinguish one element from another. A first element could be termed a second element, and, similarly, a second element could be termed a first element, without departing from the scope of the implementations of the disclosure. As used herein, the term and/or includes any and all combinations of one or more of the associated listed items.

What is claimed is:

1. An apparatus, comprising:
a first photomultiplier having:
a first side including a first array of photodiodes; and
a second side opposite the first side; and
a second photomultiplier having:
a first side including a second array of photodiodes; and
a second side opposite the first side,
the second side of the first photomultiplier being directly coupled to the second side of the second photomultiplier in a staggered arrangement.

2. The apparatus of claim 1, wherein the second side of the first photomultiplier is soldered to the second side of the second photomultiplier.

3. The apparatus of claim 1, wherein a printed circuit board is excluded from a stack including the first photomultiplier and the second photomultiplier.

4. The apparatus of claim 1, wherein the first photomultiplier includes:
at least one cathode terminal configured to be connected to external circuitry; and
at least one anode terminal configured to be connected to the external circuitry.

5. The apparatus of claim 1, wherein the first photomultiplier is included in a first wafer and the second photomultiplier is included in a second wafer, the first wafer being wafer bonded to the second wafer.

6. The apparatus of claim 1, further comprising:
a scintillator crystal coupled with the first side of the first photomultiplier, the apparatus being configured for use in a positron emission tomography (PET) scanner.

7. The apparatus of claim 6, wherein the scintillator crystal is a first scintillator crystal, the apparatus further comprising:
a second scintillator crystal coupled with the first side of the second photomultiplier.

8. The apparatus of claim 1, wherein:
the first photomultiplier includes:
a first plurality of conductive traces disposed on the second side of the first photomultiplier; and
a first plurality of through vias respectively electrically coupling a first portion of the first plurality of conductive traces with the first array of photodiodes; and
the second photomultiplier includes:
a second plurality of conductive traces disposed on the second side of the second photomultiplier; and
a second plurality of through vias respectively electrically coupling a first portion of the second plurality of conductive traces with the second array of photodiodes.

9. The apparatus of claim 8, wherein:
a second portion of the first plurality of conductive traces are respectively electrically coupled with the first portion of the second plurality of conductive traces; and
a second portion of the second plurality of conductive traces are respectively electrically coupled with the first portion of the first plurality of conductive traces.

10. The apparatus of claim 1, further comprising a flex connector coupled with the first photomultiplier, the flex connector being configured to connect the first photomultiplier with external circuitry.

11. The apparatus of claim 1, wherein:
the first photomultiplier includes:
a first semiconductor die including the first array of photodiodes; and
a first glass cover disposed on the first array of photodiodes; and
the second photomultiplier includes:
a second semiconductor die including the second array of photodiodes; and
a second glass cover disposed on the second array of photodiodes.

12. The apparatus of claim 1, wherein the first photomultiplier and the second photomultiplier each exclude a glass cover.

13. An apparatus comprising:
a scintillator crystal;
a semiconductor die including a first side and a second side opposite the first side, the semiconductor die including a photomultiplier array disposed on the first side, the scintillator crystal being disposed on the first side of the semiconductor die;
a carrier disposed on the second side of the semiconductor die, the photomultiplier array being electrically coupled with the carrier; and
a molding material disposed on a sidewall defined by at least one of the semiconductor die or the carrier, the molding material configured to protect the photomultiplier array from moisture ingress.

14. The apparatus of claim 13, wherein the carrier is electrically coupled with the photomultiplier array by at least one via that extends through the semiconductor die from the first side of the semiconductor die to the second side of the semiconductor die.

15. The apparatus of claim 13, wherein the carrier is electrically coupled with the photomultiplier array by at least one wire bond encapsulated in the molding material.

16. The apparatus of claim 13, further comprising a flex connector coupled with the carrier, the flex connector being configured to connect the photomultiplier array with external circuitry.

17. The apparatus of claim 13, wherein the molding material includes at least one of epoxy or silicone.

18. The apparatus of claim 13, wherein the molding material extends over at least a portion of the second side of the semiconductor die.

19. The apparatus of claim 13, wherein:
a perimeter portion of the carrier is partially etched; and
the molding material extends over the partially etched perimeter portion of the carrier.

20. An apparatus comprising:
a first semiconductor die including a first photomultiplier array;
a second semiconductor die including a second photomultiplier array, the second semiconductor die being coupled in a stack with the first semiconductor die such that the first photomultiplier array is disposed at a proximal end of the stack and the second photomultiplier array is disposed at a distal end of the stack;
a first scintillator crystal disposed on the first semiconductor die at the proximal end of the stack;
a second scintillator crystal disposed on the second semiconductor die at the distal end of the stack, the stack being disposed between the first scintillator crystal and the second scintillator crystal; and
an encapsulation material disposed:
on a sidewall defined by the first semiconductor die and the second semiconductor die; and
between the first scintillator crystal and the second scintillator crystal.

21. The apparatus of claim 20, further comprising:
a first flex connector coupled with the first semiconductor die, the first flex connector being configured to connect the first photomultiplier array with external circuitry; and
a second flex connector coupled with the second semiconductor die, the second flex connector being configured to connect the second photomultiplier array with the external circuitry.

22. The apparatus of claim 20, wherein the first semiconductor die is directly coupled with second semiconductor die.

23. The apparatus of claim 20, wherein:
the first scintillator crystal is directly coupled with the first semiconductor die with an optically clear adhesive; and
the second scintillator crystal is directly coupled with the second semiconductor die with the optically clear adhesive.

24. An apparatus, comprising:
a scintillator crystal;
a first photomultiplier disposed on a first sidewall of the scintillator crystal;
a second photomultiplier disposed on a second sidewall of the scintillator crystal, the second sidewall being orthogonal to the first sidewall; and
a third photomultiplier disposed on a third sidewall of the scintillator crystal, the third sidewall being parallel to the first sidewall.

25. The apparatus of claim 24, further comprising at least one glass component respectively disposed between at least one of:
the first photomultiplier and the first sidewall;
the second photomultiplier and the second sidewall; or
the third photomultiplier and the third sidewall.

26. The apparatus of claim 24, wherein the second sidewall is on a side of the scintillator crystal opposite a fourth sidewall of the scintillator crystal configured to receive gamma radiation in a positron emission tomography scanner.

27. The apparatus of claim 24, wherein:
a first printed circuit board (PCB) is electrically coupled to the first photomultiplier;
a second PCB is electrically coupled to the third photomultiplier; and
the third photomultiplier is electrically coupled to the first PCB via a flex connector.

28. The apparatus of claim 24, further comprising:
a fourth photomultiplier disposed on a fourth sidewall of the scintillator crystal, the fourth sidewall being orthogonal to the first sidewall, the second sidewall and the third sidewall; and
a fifth photomultiplier disposed on a fifth sidewall of the scintillator crystal, the fifth sidewall being orthogonal to the first sidewall, the second sidewall and the third sidewall, and parallel to the fourth sidewall.

29. The apparatus of claim 24, further comprising:
a molding material encapsulating at least a portion of the scintillator crystal and being in contact with, at least, respective portions of the first photomultiplier, the second photomultiplier, and the third photomultiplier, such that at least a fourth sidewall of the scintillator crystal is exposed through the molding material.
